(19) United States
(12) Patent Application Publication
(10) Pub. No.: US 2023/0098416 A1
(43) Pub. Date: Mar. 30, 2023

(54) Sleep Monitoring System with Multiple Vibration Sensors

(71) Applicant: MAREXA OÜ, Rae Municipality (EE)

(72) Inventors: Tanel TÄTTE, Tartu (EE); Tauri TÄTTE, Räni alevik, Kambja val (EE); Glen KELP, Võru (EE); Toomas VARJUND, Kose vald (EE); Egert ISPERT, Viljan di vald (EE); Alivar LUMISTE, Viljandi vald (EE); Vladimir I. KONTRATIEV, Leningradskaya obl. (EE)

(21) Appl. No.: 17/801,392

(22) PCT Filed: Feb. 24, 2021
(86) PCT No.: PCT/EP2021/054598

§ 371 Date: Aug. 22, 2022

(30) Foreign Application Priority Data
Feb. 24, 2020 (GB) 2002573.0

(51) Int. Cl. A61B 5/00; A61B 5/0205

(52) U.S. Cl. CPC A61B 5/7264; A61B 5/0205; A61B 5/4094; A61B 5/4875; A61B 5/4809; A61B 5/746; A61B 5/7282; A61B 2562/0204; A61B 2562/0247

(57) ABSTRACT

According to an aspect of the invention there is provided a system for use in monitoring one or more physiological states of a user, the system comprising one or more processors configured to: receive a pressure signal representing pressure within a cushioning layer supporting at least a portion of a user and an acoustic signal representing acoustic vibrations within the cushioning layer; and determine, based on the pressure signal and acoustic signal, the one or more physiological states of the user.

### TECHNICAL FIELD

The present invention relates to systems and methods for monitoring one or more physiological states of a user. More specifically, embodiments relate to processing audio and pressure signals representing vibrations within a cushioning layer supporting at least a portion of a user in order to determine one or more physiological states of the user, such as body position, hydration level and level of consciousness.

### BACKGROUND

Sleep quality is of great importance to people's daily activities. Research performed by the National Sleep Foundation (USA) suggests that poor or insufficient sleep affects live of almost 45% of Americans. Many visit doctors regularly to seek improvements to the quality of their sleep. The research estimates that around 33% of the population rate their sleep as no better than fair or poor.

While it is clear that humans require sleep to function properly, the quality and quantity of sleep required is a complex problem. A great deal of research in recent years has focused on understanding sleep and its physiological and psychological effects. For example, some individuals, who sleep too little, may feel tired or fatigued during the day while others who sleep too many hours have a similar feeling of grogginess as a result of sleeping too much (see U.S. Patent Application No. 62/108,149).

Researchers continue to study many different physiological conditions during sleep to understand the complex interplay between sleep and wakeful wellbeing. To study this, different technologies have been suggested for monitoring sleep.

The monitoring of sleep is important for many reasons. Most of all, it provides actual data about sleeping—length of sleep, depth of sleep, times users go to sleep and when they wake up. Having this data in hand, people with sleep problems are motivated (or may be prompted) to visit a sleep specialist to seek treatment.

Physiological parameters, such as heart rate, can be measured using wearable electronics; however, this can be uncomfortable for the user to sleep in, thereby negatively affecting the user's quality of sleep.

In light of the above, there is a need for an improved means of monitoring sleep that is accurate and has improved comfort for the user.

### SUMMARY

The present invention seeks to provide a system for use in monitoring one or more physiological states of a user.

According to an aspect of the invention there is provided a system for use in monitoring one or more physiological states of a user, the system comprising one or more processors configured to: receive a pressure signal representing pressure within a cushioning layer supporting at least a portion of a user and an acoustic signal representing acoustic vibrations within the cushioning layer; and determine, based on the pressure signal and acoustic signal, the one or more physiological states of the user.

By making use of pressure and acoustic signals representing vibrations within a cushioning layer, the present disclosure allows one or more physiological states to be more effectively determined whilst the user is sitting or lying upon the cushioning layer. The cushioning layer may form part of a bed, chair or other type of furniture. Accordingly, the user can be monitored non-invasively through monitoring the natural vibrations within the cushioning layer caused by the user's movements (e.g. respiration, heartbeat and body movement).

The pressure signal may represent pressure oscillations at a lower frequency than the acoustic vibrations. This allows isolation between the two pressure signals and acoustic signals in the frequency domain, which helps minimise mutual interference the two signals. The separations of the two signals in the frequency allows the two signals to be independently processed without the need for further signal processing, although further signal processing (such as filtering) may be used.

The one or more physiological states of the user may comprise one or more of the following: a body position of the user; a level of hydration of the user; and one or more abnormal physiological states of the user.

The pressure signal may comprise a respiration signal indicative of respiration of the user and the determination of the one or more physiological states of the user may be based on the respiration signal. That is, at least a component of the pressure signal may be the respiration signal. By monitoring the respiration signal, various physiological parameters may be determined (such as respiration rate).

The one or more physiological states of the user may comprise one or more abnormal physiological states of the user determined based on changes in the respiration signal, the one or more abnormal physiological states of the user may include one or more of the following: an interruption in breathing; and an irregularity in breathing pattern. Determination of an interruption in breathing may be determination of sleep apnea, although an interruption in breathing need not be linked to the user sleeping.

An interruption in breathing may be determined in response to a detection of an absence of change in the respiration signal for a predetermined duration. The respiration signal may have an amplitude present during an interruption in breathing.

Accordingly, detecting for an absence of change in the respiration signal improves the accuracy in determination of an interruption in breathing of the user.

An irregularity in breathing may be determined in response to a detection of irregular changes in respiration rate of the respiration signal. This may either be through determination of respiration rate and detection of a greater than threshold change or variability in respiration rate. Alternatively, this may be through other monitoring techniques, such as the application of a machine learning classifier (as discussed later).

The acoustic signal may comprise a cardiac signal indicative of one or more cardiac cycles of the user and wherein the determination of the one or more physiological states of the user may be based on the cardiac signal. That is, at least a component of the acoustic signal may be the cardiac signal. By monitoring the cardiac signal, various physiological parameters may be determined (such as heartrate).

The one or more physiological states of the user may comprise one or more abnormal physiological states of the user determined based on changes in the cardiac signal, wherein the one or more abnormal physiological states may include one or more of the following: an interruption in heartbeat; an irregularity in heartbeat; and presence of one or more abnormal sounds synchronous with heartbeat of the user.

An interruption in heartbeat may be determined in response to a detection of an absence of change in the cardiac signal for a predetermined duration. The respiration signal may have an amplitude present during an interruption in heartbeat. Accordingly, detecting for an absence of change in the cardiac signal improves the accuracy in determination of an interruption in heartbeat of the user.

An irregularity in heartbeat may be determined in response to a detection of irregular changes in heart rate as shown in the cardiac signal. Determination of an irregularity in heartbeat may include a determination of heart arrhythmia.

Changes in heart rate may be shown by changes in frequency of cardiac cycles in the cardiac signal.

The presence of one or more abnormal sounds synchronous with heartbeat may be determined using a machine learning classifier. Alternatively, the presence of one or more abnormal sounds synchronous with heartbeat may be determined in response to a detection that an amplitude of the acoustic signal or cardiac signal repeatedly exceeds a predefined threshold at a same point within multiple cardiac cycles within the cardiac signal. The same point within multiple cardiac cycles need not be exactly the same, but could be within a given range. The same point within multiple cardiac cycles may be determined by determining whether the amplitude is exceeded within a same phase range across the multiple cardiac cycles.

The pressure signal may comprise a respiration signal indicative of respiration of the user and the acoustic signal may comprise a cardiac signal indicative of one or more cardiac cycles of the user and wherein the determination of the one or more physiological states of the user may be based on the respiration signal and the cardiac signal.

The one or more physiological states of the user may comprise one or more abnormal physiological states of the user determined based on changes in the pressure signal and acoustic signal, wherein the one or more abnormal physiological states may include one or more of the following: presence of one or more abnormal sounds synchronous with breathing; death; seizure; and bruxism.

The presence of one or more abnormal sounds synchronous with breathing may be determined using a machine learning classifier. Alternatively, the presence of one or more abnormal sounds synchronous with breathing may be determined in response to detecting that an amplitude of the acoustic signal or cardiac signal repeatedly exceeds a predefined threshold at a same point within multiple respiratory cycles within the respiration signal. One or more abnormal sound synchronous with breathing may be snoring. The same point within multiple respiratory cycles need not be exactly the same point, but could be within a given range. The same point within multiple respiratory cycles may be determined by determining whether the amplitude is exceeded within a same phase range across the multiple respiratory cycles.

Death may be determined in response to a detection of an absence of the cardiac signal or an absence of changes in the cardiac signal whilst the respiration signal continues to be detected or changes in the respiration signal continue to be detected for a predefined time interval.

Seizure may be determined in response to a detection that the pressure signal or respiration signal exceeds an amplitude threshold and a frequency of the acoustic signal or cardiac signal exceeds a frequency threshold.

Determining the one or more physiological states of the user may comprise: obtaining an input derived from one or both of the pressure signal and acoustic signal; inputting the input into a machine learning model configured to determine, for each of a set of potential physiological states, a probability that the user has the corresponding physiological state; determining the one or more physiological states based each probability output from the machine learning model.

The machine learning model may be a neural network. The neural network may comprise a series of layers, each comprising nodes with corresponding weights. The neural network may be represented by a set of parameters (e.g. the weights of the nodes). The neural network may be trained to estimate the probability of each of the set of potential physiological states through training based on a training data set comprising labelled training data. Training might involve updating the parameters of the neural network to reduce an error of the output of neural network relative to the labelled (ground truth) training data.

Bruxism may be determined using the machine learning model, wherein the model is a classifier configured to detect noise in the pressure signal or respiration signal indicating movement of the jaw and noise in the acoustic signal or cardiac signal indicating teeth grinding.

The one or more physiological states of the user may comprise a body position of the user detected based on a relative phase of the pressure signal to the acoustic signal.

The one or more processors may be further configured to detect a cardiac pressure signal of the user in the pressure signal and the determination of a body position of the user may be based on the cardiac pressure signal.

The cardiac pressure signal is different to the cardiac signal in that the cardiac pressure signal is detected from the pressure signal, whereas the cardiac signal is contained within the acoustic signal. Accordingly, the cardiac pressure signal and cardiac signal may both be used to determine body position.

The cardiac pressure signal may be detected over a measurement window starting at a start measurement time and ending at an end measurement time, wherein the start measurement time may be triggered by a first feature in the cardiac signal representing a start of the systolic phase of the cardiac signal and the end measurement time may be triggered by a second feature in the cardiac signal representing an end of the systolic phase of the cardiac signal.

By windowing the cardiac pressure signal, noise can be effectively filtered out to ensure that only pressure fluctuations due to heart movements are considered. This also allows the systolic phase of the cardiac cycle to be detected using the acoustic signal, which will be a more accurate indicator of the various heart phases.

The determination of the body position of the user may be based on a phase of the cardiac pressure signal during the systolic phase of a cardiac cycle shown within the cardiac pressure signal.

The phase of the cardiac pressure signal during the measurement window may correspond to the body position of the user such that the user lying on one side may correspond to a phase of the cardiac pressure signal where the amplitude is at a local maximum during the systolic phase and the user lying on an opposite side may correspond to another phase of the cardiac pressure signal where the amplitude is at a local minimum during the systolic phase.

The determination of the body position of the user may additionally be based on a relative amplitude of the cardiac pressure signal. The relative amplitude of the cardiac pressure signal during the measurement window may correspond to a distance of the heart from the cushioning layer of the user.

The one or more processors may be further configured to output an alert if the system determines that the body position of the user has remained unchanged for a predetermined duration of time.

The one or more physiological states of the user may comprise a level of hydration of the user.

The level of hydration of the user may be determined by: detecting, for each cardiac cycle in the cardiac signal, a peak of the cardiac signal across a cardiac cycle; determining across a respiratory cycle comprising a plurality of cardiac cycles, a maximum of the peaks for the plurality of cardiac cycles and a minimum of the peaks for the plurality of cardiac cycles; determining a difference between the maximum and the minimum; determining an overall amplitude of the cardiac signal across the respiratory cycle; and determining a ratio of the difference to the overall amplitude as an indicator of the level of hydration.

For users with heart failure, the level of hydration may be considered optimal if the ratio of the difference is less than 10%. For users that accumulate water in the lungs or other organs, the level of hydration may be considered optimal if the ratio of the difference is less than 20%

The one or more processors may be further configured to output an alert if the system determines that the level of hydration of the user has exceeded a predetermined hydration level threshold.

The one or more processors may be further configured to output a control signal to control an external device based on the user's physiological state.

The one or more physiological states may comprise a level of consciousness of the user and the one or more processors may be configured to output the control signal in response to determining that the level of consciousness falls within a threshold range.

According to another aspect of the invention there is provided a method for determining one or more physiological states of a user, the method comprising: receiving a pressure signal representing pressure within a cushioning layer supporting at least a portion of a user and an acoustic signal representing acoustic vibrations within the cushioning layer; and determining, based on the pressure signal and acoustic signal, the one or more physiological states of the user.

According to further aspect of the invention there is provided a non-transitory computer-readable medium containing programming instructions that, when executed by a computer, cause the computer to: receive a pressure signal representing pressure within a cushioning layer supporting at least a portion of a user and an acoustic signal representing acoustic vibrations within the cushioning layer; and determine based on the pressure signal and acoustic signal, the one or more physiological states of the user.

### BRIEF DESCRIPTION OF THE DRAWINGS

Arrangements of the present invention will be understood and appreciated more fully from the following detailed description, made by way of example only and taken in conjunction with drawings in which:

FIG. 1 shows a cross sectional view and a zoomed in cross sectional view of a sleep monitoring apparatus according to an embodiment;

FIG. 2 shows a cross sectional view of the sleep monitoring apparatus embedded within a cushioning layer;

FIG. 3 shows a sleep monitoring apparatus comprising a network of tubes according to an embodiment of the invention;

FIG. 4 shows a sleep monitoring apparatus according to an embodiment embedded within a cushioning layer forming part of a bed;

FIG. 5 shows a side view of the sleep monitoring apparatus of FIG. 4;

FIG. 6 shows an alternative embodiment in which tubes extend longitudinally down the length of a bed;

FIG. 7 shows a perspective view of the embodiment of FIG. 6;

FIG. 8 shows a typical graphical representation of a pressure signal detected by a sensing unit according to an embodiment;

FIG. 9 shows an intensity against time graph of the signal retrieved by the sensing unit in relation to heartbeat and breathing;

FIG. 10 shows a cross-sectional view of a sleep monitoring apparatus according to an embodiment of the invention;

FIGS. 11A and 11B show how the sleep monitoring apparatus may be arranged inside a mattress according to an embodiment of the invention;

FIG. 12 shows a side view of the sleep monitoring system of FIG. 11A;

FIGS. 13A-13F show examples of different cross sections for the tube according to various embodiments;

FIG. 14 shows the sensing unit according to an embodiment;

FIG. 15 shows a manufacturing process for the integration of the sleep monitoring apparatus into a cushioning layer;

FIG. 16 shows a table detailing a method for determining a sleep level of the user according to an embodiment;

FIG. 17 shows a flow chart for a method of determining when to activate an alarm based on determined sleep levels according to an embodiment;

FIG. 18 shows a pressure signal representing pressure within a cushioning layer supporting at least a portion of a user and an acoustic signal representing acoustic vibrations within the cushioning layer;

FIG. 19 shows a graphical representation of pressure and acoustic signals showing an interruption in breathing of the user;

FIG. 20 shows a graphical representation of pressure and acoustic signals showing an irregularity in breathing of the user;

FIG. 21 shows a graphical representation of pressure and acoustic signals showing an interruption in heartbeat of the user;

FIG. 22 shows a graphical representation of pressure and acoustic signals showing an irregularity in heartbeat of the user;

FIG. 23 shows a graphical representation of abnormal sounds synchronous with heartbeat of the user;

FIG. 24 shows a graphical representation of pressure and acoustic signals showing abnormal sounds synchronous with breathing of the user;

FIG. 25 shows another graphical representation of pressure and acoustic signals showing abnormal sounds synchronous with breathing of the user, wherein the abnormal sounds are snoring;

FIG. 26 shows a graphical representation of pressure and acoustic signals showing death of the user;

FIG. 27 shows a graphical representation of pressure and acoustic signals showing a seizure of the user;

FIG. 28A and FIG. 28B show graphical representations of pressure and acoustic signals indicative of body positions of the user;

FIG. 29 shows the sleep monitoring apparatus being used in a multiple user operation mode;

FIG. 30 shows a plan view of the sleep monitoring apparatus being used in a multiple user operation mode;

FIG. 31 shows a plot of level of consciousness over time, divided into various sleep states; and

FIG. 32 shows a graphical representation of pressure and acoustic signals indicative of the user's level of hydration.

### DETAILED DESCRIPTION

According to a first aspect there is provided a system for use in monitoring one or more physiological parameters of a user. The system comprises: at least one sensing unit comprising at least one vibration sensor; and at least one elastic tube configured to be embedded within a cushioning layer for supporting at least a portion of the user. One end of the at least one tube is sealed and the other end of the at least one tube is closed by the at least one sensing unit so as to form a volume that is filled with fluid and that is defined by one or more inner walls of the tube and a surface of the at least one sensing unit. The at least one elastic tube is configured to transmit vibrations from one or more sections of the at least one tube to the at least one sensing unit for detection by the at least one vibration sensor.

By providing an elastic tube for transferring vibrations, the sensing unit may be located away from the user's body thereby avoiding damage to the sensing unit by mechanical strain in the cushioning layer and also avoiding the sensing unit impacting the comfort of the cushioning layer. By making the tube from an elastic material, mechanical vibrations are more effectively transferred, whilst also allowing the tube to return to its original shape once pressure has been released to allow the tube to continue functioning.

The tube need not be cylindrical, but may be any elongate hollow structure. The tube may be straight or may comprise one or more bends along its length. The fluid may be a gas or a liquid. The fluid might be air or any other gas capable to transmitting vibrations. The tube may be made from elastic material such as rubber (e.g. natural rubber, synthetic rubber, silicone rubber, etc.), urethane, or polydimethylsiloxane.

According to an embodiment, the at least one vibration sensor comprises one or more of a microphone and a pressure sensor. The at least one vibration sensor may operate at a frequency capture range from 0-200 Hz. That is, the microphone and/or the pressure sensor may operate at a frequency capture range from 0-200 Hz.

According to an embodiment, the at least one elastic tube has a uniform cross-section along its length. This helps to avoid reflections within the tube.

According to an embodiment, the at least one elastic tube comprises one or more indents along the one or more inner walls of the tube to help prevent the tube sticking shut when crushed. The one or more indents may be in the form of one or more ridges running along the length of the tube. The one or more indents may comprise a plurality of evenly spaced indents around the circumference of the tube. This helps to prevent sticking even when crushed from a variety of angles.

According to an embodiment, the at least one elastic tube comprises a plurality of elastic tubes that are connected to a common node to form a single volume for transmitting vibrations back to the sensing unit. This allows a larger volume of the cushioning layer to be monitored.

According to an embodiment, the system further comprises a controller configured to determine one or more of: one or more physiological parameters of the user from detected vibrations; and one or more physiological states of the user from detected vibrations.

In one embodiment, the controller forms part of an external device and wherein the at least one sensing unit is configured to send data relating to the detected vibrations to the external device for processing by the controller. This might be a computer, a server, a mobile device, or any form of device with processing capabilities.

In an alternative embodiment, the controller forms part of the sensing unit.

The at least one vibration sensor may be configured to generate a vibration signal (a data transmission, such as an electrical signal, indicative of the amplitude of the detected vibrations) and send this vibration signal to the controller. This vibration signal may be analogue or digital. Where the controller forms part of an external device, the sensing unit might transmit this vibration signal to the controller, or might process the vibration signal further (e.g. via compression) before transmitting the processed signal to the controller.

According to an embodiment, the controller is configured to determine, based on the detected vibrations, one or more of a heart rate of the user, a respiration rate of the user, a body position of the user and movement of the user.

According to one embodiment, the controller is configured to determine the heart rate of the user. Determining the heart rate may comprise: filtering the detected vibrations to form filtered vibration data over a predefined frequency range; detecting local maxima in the filtered vibration data; and determining the heart rate based on the detected local maxima. The predefined frequency range may be a range that maintains a large proportion of the heartbeat information whilst filtering out noise and respiration rate information. In one embodiment, the predefined frequency range is 10 to 30 Hz.

According to one embodiment, the controller is configured to determine the respiration rate of the user, wherein determining the respiration rate comprises: detecting local maxima in the detected vibrations; and determining the respiratory rate based on the detected local maxima. The respiratory rate may be determined over different frequency range to the heart rate (e.g. over a range of 0-200 Hz rather than 10-30 Hz)

According to an embodiment, the controller is configured to determine movement of the user based on the detected vibrations, wherein determining movement of the user comprises determining whether an amplitude of the detected vibrations exceeds a predefined amplitude threshold.

According to an embodiment, the at least one vibrations sensor comprises a microphone configured to generate a microphone signal based on the detected vibrations and a pressure sensor configured to generate a pressure sensor signal based on the detected vibrations. The controller is configured to determine the body position of the user, wherein the body position of the user is determined based on phase differences in the microphone and pressure sensor signals.

The determined body position might indicate whether the person is: on their back, on their stomach, on their left or on their right side.

According to one embodiment, the controller is further configured to determine the physiological state (e.g. respiration rate and/or sleep level) of the user based on specific repetitive noises within the detected vibration (e.g. within a detected respiration cycle).

According to an embodiment, the system is configured to export or store data relating to the detected vibrations (e.g. a raw vibration signal) only when certain conditions are met. These conditions may include one or more of:
(1) the sleep state of the user (e.g. the user being in deep sleep);
(2) no movements being detected within in a predefined preceding period of time (e.g. the last five minutes);
(3) the variability of the vibrations within a predefined preceding period of time (e.g. a moving average root-mean-square (RMS) value of the signal has not changed by more than a predefined amount over a predetermined period of time (e.g. 20% in the last five minutes)); and
(4) the system has detected a heartbeat or a breathing cycle (e.g. heart rate and breathing rate are able to be determined from the vibration signal).

According to an embodiment, the controller is configured to determine a sleep state of the user based on one or more of the heart rate of the user, the respiration rate of the user and movement of the user. Sleep state may be one of awake, light sleep, deep sleep and rapid eye movement (REM) sleep.

According to an embodiment, the controller is configured to perform one or more of the following: determine that the user is awake in response to determining that one or both of the heart rate and respiration rate are above a corresponding first threshold and variation in one or both of the heart rate and respiration rate is below a corresponding variation threshold; determine that the user is in rapid eye movement, hereinafter referred to as REM, sleep in response to determining that one or both of the heart rate and respiration rate are above the corresponding first threshold and the variation in one or both of the heart rate and respiration rate is above the corresponding variation threshold; determine that the user is in light sleep in response to determining that one or both of the heart rate and respiration rate are below the corresponding first threshold and above a corresponding second threshold that is less than the corresponding first threshold; and determine that the user is in deep sleep in response to determining that one or both of the heart rate and respiration rate are below the corresponding second threshold.

A corresponding first/second threshold means different first thresholds or different second thresholds may be set for heartrate and respiration rate.

According to an embodiment, the system further comprises an alarm system that is configured to issue an alarm in response to a determination that a predefined sleep state or predefined change in sleep state has been reached during an alarm period between a predefined start alarm time and a predefined end alarm time. The alarm system may be integrated within the sensing unit (i.e. in the form of a controller in the sensing unit) or may be an external device to the sensing unit (that need not be the same external device as discussed above with regard to the determination to physiological parameters).

According to an embodiment, the alarm system is configured to perform one or more of the following: issue the alarm in response to a determination that the user has transitioned from REM sleep to light sleep during the alarm period; issue the alarm in response to a determination that a current time is within a predefined period from an end alarm time and the user is in light sleep during the alarm period, the predefined period being shorter than the alarm period; and issue an alarm in response to the end alarm time being reached and the predefined sleep state or predefined change in sleep state has not been detected during the alarm period.

According to a further aspect there is provided a computer system comprising one or more processors configured to: receive one or more vibration signals indicative of vibrations detected within a cushioning layer for supporting at least a portion of the user; and determine, based on the one or more vibration signals, one or more of a heart rate of the user, a respiration rate of the user, a body position of the user and movement of the user.

According to an embodiment, the one or more processors are further configured to determine a sleep state of the user based on one or more of the heart rate of the user, the respiration rate of the user and movement of the user.

According to an embodiment, the one or more processors are further configured to issue an alarm in response to a determination that a predefined sleep state or predefined change in sleep state has been reached during an alarm period between a predefined start alarm time and a predefined end alarm time.

According to a further aspect there is provided a kit of parts for use in monitoring one or more physiological parameters of a user. The kit of parts comprises: at least one sensing unit comprising at least one vibration sensor; and at least one elastic tube configured to be embedded within a cushioning layer for supporting at least a portion of the user, wherein one end of the at least one tube is sealed and the other end of the at least one elastic tube open for receiving the at least one sensing unit. The kit of parts is configured such that when the at least one sensing unit is received within the other end of the at least one elastic tube, a volume is defined by one or more inner walls of the tube and a surface of the at least one sensing unit for containing fluid such that the at least one elastic tube is configured to transmit vibrations from one or more sections of the at least one tube to the at least one sensing unit for detection by the at least one vibration sensor.

Embodiments as described herein may be implemented as devices, systems, kits of parts, methods or non-transitory computer readable media.

Embodiments described herein relate to systems for monitoring physiological parameters of users in bed and, in particular, of monitoring sleep.

In order to avoid requiring a user to wear, or be connected to, any monitoring system(s), the present embodiments integrate sensors into the bed itself. This provides a less intrusive means of monitoring users' sleep and avoids causing discomfort to the user through the application of wearable technology. In addition, as the sensors are integrated into the bed, the sensors can be provided with a more reliable power supply, either by sourcing power from the mains electricity supply (grid power), or by providing a larger battery than would otherwise be feasible on a wearable device due to size and comfort constraints. This reduces the risk of measurements being missed due to loss of power, which can be problematic due to the relatively long period of sensing and the fact that the users will usually be unaware that power has been lost, as they are asleep.

In general, there have been proposed two different approaches for monitoring sleep of a person on a bed. One of these is based on measuring sleep rhythms through sensors that are separated from the bed and the person being monitored, such as acoustic noise sensors or optical cameras. The other option is to place motion sensitive elements directly into contact with the body, or very close to body of the person being monitored. For the latter, sensors are placed, for example, under the sleeping lever of the mattress.

The drawback of the first solution—monitoring of sleeping person over distance—is its inexactness. It is difficult to obtain accurate measurements when the sensors are not located close to the person being sensed. The second method—placing sensors close to the body—enables more exact measurements, for example, the monitoring of even heart rhythms and breathing of a sleeping person. Having said this, placing sensors in close proximity to the body of the person being measured (e.g. directly under the sleeping surface) is problematic because they can easily break under mechanical stress caused by the persons on the bed. This is especially critical when long-term guaranties for the system may be requested. Adapting sensors to withstand the additional level of stress can greatly increase the complexity and cost of the sensors. Furthermore, integrating such sensors directly into the mattress is likely to negatively affect the comfort of the mattress.

Data on sleep can not only help to identify medical conditions, but it can also be used to control other user devices that might disturb the user during sleep. For example, some devices disturb sleep by causing noise or light when the user is asleep, or in the run-up to the user's bedtime. By obtaining high quality sleep data, smart devices can be controlled to improve the environment of the bedroom to optimise it in order to improve the user's sleep. For example, devices that activate motors or actuators would usually cause noise during this process. Therefore, the user's sleep can be improved if such devices are controlled so that they are switched on only when no one is sleeping in bed or only when user(s) are sleeping deeply (and therefore unlikely to be woken).

One of the example of problems where such smart switch-on-and-off devices are needed is related to poor sleep quality caused by dustiness of the air in the bedroom. People who have dust allergies are familiar with sneezing and other uncomfortable symptoms. Dust allergies also cause congestion (leading to breathing difficulties), or cause their eyes to itch or become red and watery. Approximately 10% of the population are allergic to air dusts, such as: pollens, molds, pet hairs, dust mites, etc. These symptoms effectively lower sleep quality. Therefore, air filtering that enables removal of dust particles, which are causing these problems, would enable to increase sleep quality largely. Unfortunately, air filtering for dust removal causes mechanical noise, which affect sleep quality negatively.

Due to the above-mentioned problems, there is a need for improved means of monitoring physiological parameters and conditions, and in particular sleep, in a more accurate and less intrusive manner.

Whilst the above issues are discussed with regard to monitoring sleep on a mattress, similar issues can be encountered when attempting to monitor physiological condition (e.g. heart rate, breathing rate, stress, etc.) via integration of sensing technology into other types of furniture, such as seating (armchair, sofa, etc). For instance, sensors may be integrated into the cushion or back support of a chair or other form of seat, to monitor physiological variables, such as breathing and heartrate, and physiological condition, such as stress. Having said this, direct integration of sensors close to the supporting surface of the seat can cause discomfort and increase mechanical stress upon the sensors.

The embodiments described herein aim to provide a solution for the problems mentioned above. Instead of placing sensors directly into the mattress (under the sleeping surface or into the seat under the seating surface), the present application proposes to lead signals (e.g. mechanical vibrations) out from the bed or seat, with the help of gaseous fluidic vibration conducting medium based collectors (conductors of mechanical vibrations) which we call signal collectors and which are placed under the sleeping/seating surface.

A signal collector is an elastic vessel filled with a vibration conducting fluidic, and preferably gaseous, medium, such as air. It can be made from a polymeric material, for example, rubber or PDMS. It can be a pipe that encloses a fluid (such as a gas or liquid) inside as a vibration conducting medium.

The system can make use of a gas or a liquid as vibration conducting mediums. A gas-based medium has advantages over a liquid-based medium, as liquid-based systems would be technically more problematic due to their lower durability, and the risk of the liquid evaporating over time if the signal collector is not effectively sealed. Signals, caused by outer force (e.g. a person on the bed) on the surface of collector are conducted by the fluid filling the reservoir (hollow region of the collector) and lead out from the mattress along the elongated collector, where they are detected by, for example, by pressure sensors. The signal collectors are formed of elastic material as this helps to transfer external mechanical vibrations into the conducting fluidic medium.

The signal collector can be used to lead mechanical vibrations, caused by a person sleeping on the bed, out from the mattress. When the signals are conducted out from the mattress, it is simple to connect sensors to the signal collector to measure the mechanical vibrations or pressure changes. This allows measurements to be taken outside of the mattress or at least at a distance from the human body so as to avoid mechanical stress on the sensors and to avoid negatively affective the comfort of the mattress. This configuration has many advantages compared to placing sensors directly under the sleeping surface:
Locating the sensors outside of the mattress, or at the periphery of the mattress, makes it easier to connect the signal collector to the sensors
The mechanical stress on the sensors is reduced, thereby reducing the risk of damage and allowing for the use of less durable (and therefore less expensive) sensors
The solution is cost effective a single sensor can sense signals over a large sensing area via the use of the collector system spread under the sleeping surface.

Measuring of signals with the help of the collector placed between the signal source and sensor might result in some losses of signal quality and quantity. However, recent enhancements in sensor quality mean that sensors are often far more sensitive than needed for the purpose of sleep monitoring, including for example: acceleration sensors, pressure sensors, optical sensors, (optical) position sensors, etc. Still, signal collectors could also force small vibrations caused by a stronger force, by transferring the force into more intense mechanical motions before measurement.

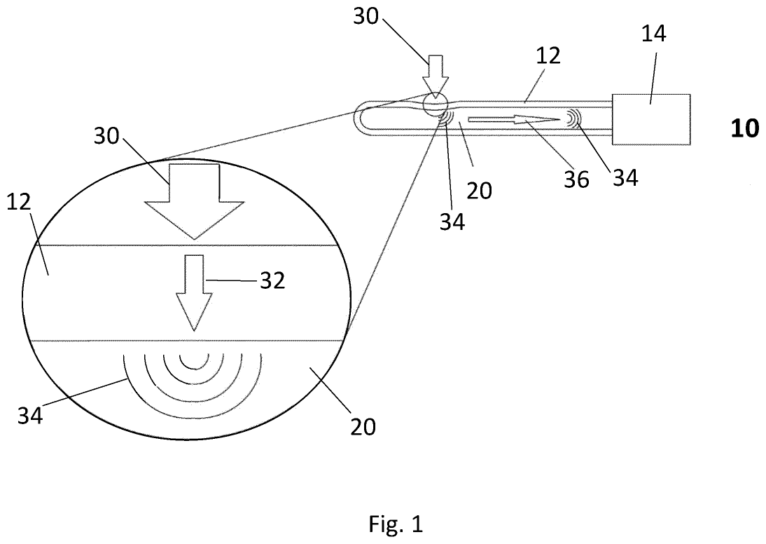

FIG. 1 shows a cross sectional view and a zoomed in cross sectional view of a sleep monitoring apparatus 10 according to an embodiment. The sleep monitoring apparatus 10 comprises a tube 12 and a sensing unit 14 containing fluid 20.

The tube 12 forms a signal collector for transmitting mechanical vibrations to the sensing unit 14. The tube 12 is elongated along a longitudinal axis. The tube 12 is closed on one end (the distal end) by the tube itself to hold the fluid 20, whilst the other end (the proximal end) opens onto the sensing unit 14 via a coupling section of the tube 12. The coupling section 12 is configured to couple with the sensing unit 14 so that the sensing unit 14 closes the open, proximal end of the tube 12 thereby containing the fluid 20 within an internal volume of the tube 12 (defined by inner walls of the tube 12). The sensing unit 14 may be coupled to the proximal end of the tube 12 via a sealing portion, locking mechanism or any other suitable means to prevent the fluid 20 from escaping the tube 12.

The fluid 20 may be a gas or liquid. Preferably, the fluid 20 is air.

The tube 12 is formed of a flexible material to enable the effective transfer of external pressure to the fluid 20. Preferably, the tube 12 is elastic. The tube may be made from materials such as rubber or polydimethylsiloxane (PDMS) or any other material that would be suitable to transmit the external force 30 as useful signals to the sensing unit 14. It is important that the tube 12 retains its initial size and shape as soon the external force is eliminated. Forming the tube 12 from elastic material provides resilience such that the tube 12 returns to its original shape upon removal of the force.

The sensing unit 14 comprises a pressure sensor, which detects pressure changes in the fluid 20. When an external force 30 is applied to the tube 12, the external force is transmitted to the fluid 20 through deformation of the wall of the tube 12. The external force 30 creates a vibration 34 inside the fluid, specifically a pressure change in the fluid 20. This vibration 34 is propagated along the length of the tube 12 until it reaches the proximal end of the tube 12 at which the sensing unit 14 is located. The sensing unit 14 then senses the vibration 34.

In one embodiment, the sensing unit 14 comprises a controller configured to determine physiological parameters from the sensed vibrations. In another embodiment, the sensing unit 14 comprises an output module (such as a wireless transmitter) for sending vibration measurements to an external system that is configured to determine physiological parameters from the sensed vibrations.

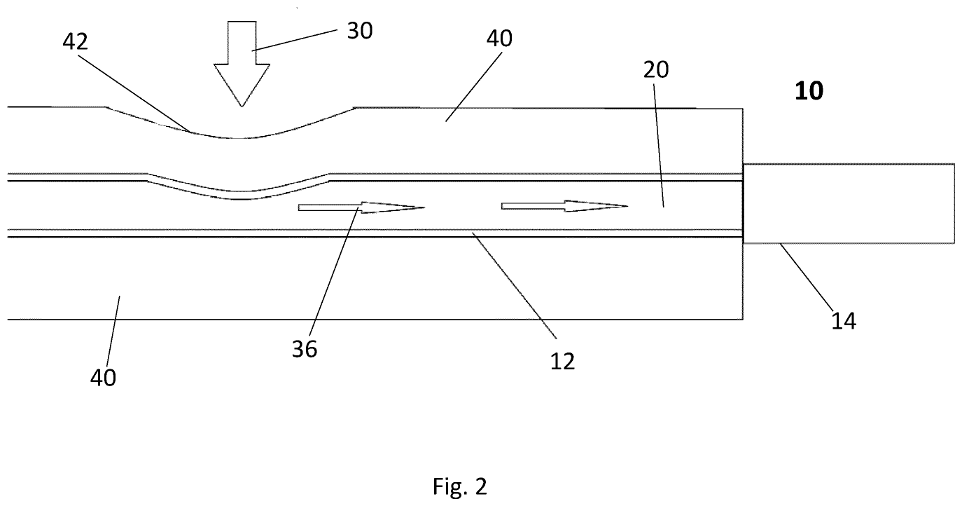

FIG. 2 shows a cross sectional view of the sleep monitoring apparatus 10 embedded within a cushioning layer 40. The sleep monitoring apparatus 10 is situated inside the cushioning layer 40 and underneath a contact surface 42. The cushioning layer 40 is a deformable and resilient layer for supporting a portion of a human body. The cushioning layer may be filled within cushioning material, may be filled with a cushioning fluid (such as air) and/or may comprise one or more elastic sheets or threads for providing support. The cushioning layer 40 may form of be part of, for instance, a mattress, or mattress topper within a bed or a cushion or back support of a chair. The contact surface 42 is a surface configured to receive pressure from a portion of the human body, in use.

When an external force 30 is applied to the contact surface 42 of the cushioning layer 40, the external force 30 is transmitted to the walls of the tube 12. Consequently, pressure change is induced in the fluid 20 due to the vibrations 34 of the tube 12 caused by the external force 30.

In the present embodiment, the sensing unit 14 is situated outside of the cushioning layer 40; however, as shall be described below, it may also be embedded within the cushioning layer 40. Ideally, the sensing unit 14 away from a supporting section of the cushioning layer 40 that is configured to receive the weight of the user. For instance, the sensing unit 14 may be located at a periphery of the cushioning layer 40.

Nevertheless, the tube 12 is located under the supporting section to transfer vibrations to the sensing unit 14. In one embodiment, the sensing unit 14 is be placed at least 5 cm or more away from the supporting section.

The cushioning layer 40 can be (or form part of), but is not limited to: a mattress, a seat, a cushion or any other object configured to support or be urged against a significant or substantial part of a person's body for a prolonged duration of time. Ideally, the cushioning layer will be configured to support or be urged against a torso of the user, to aid in the detection of breathing rate and heart rate.

The contact surface 42 (or supporting surface) is any part of the surface of cushioning layer 40 that can be, but is not limited to: a sleeping surface, a seating surface, a lying surface, a resting surface or any other surface that serves to be in contact with a significant or substantial part of a person's body for a prolonged duration of time.

From the signals received by the sensing unit 14 from the vibrations 34 of the tube 12 due to the external force 30, the sleep monitoring apparatus 10 may determine physiological properties of the person in relation to sleep, such as body movements, body position (such as determination as to whether the person is lying on back, side or stomach), breathing rate and heart rate. Alternatively, measurements (or derived physiological parameters) may be exported (e.g. via a wireless connection) to an external system configured to determine these physiological properties.

Whilst the embodiment of FIG. 1 relates to a collector comprising a single longitudinal tube 12, the collector might be formed of a variety of shapes.

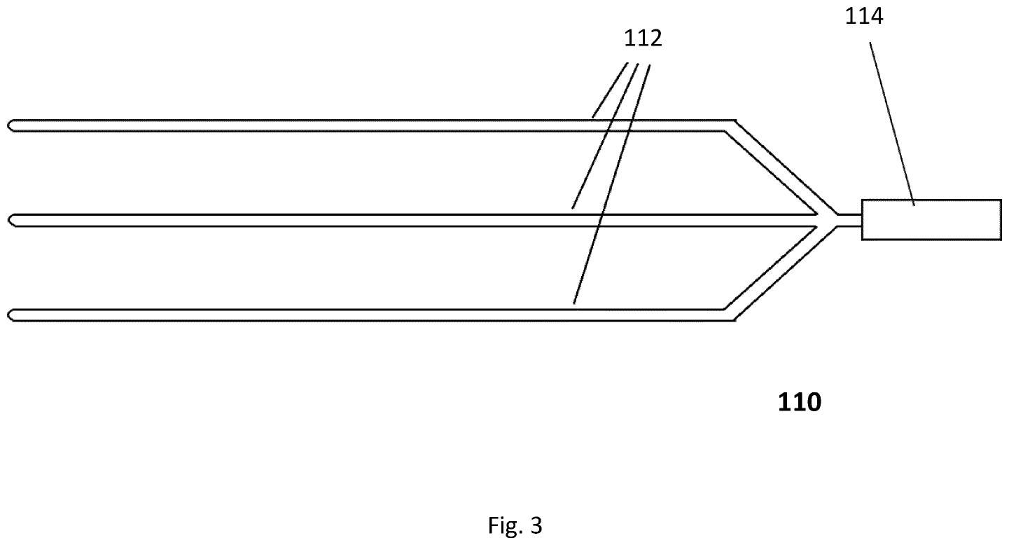

FIG. 3 shows a sleep monitoring apparatus 110 comprising a network of tubes 112 according to an embodiment of the invention. Three separate tubes 112 form a collector that is able to pick up and transfer vibrations from a larger area. The three tubes 112 are connected in parallel to a common node that is connected to the sensing unit 114 such that the network of tubes 112 are connected to each other to form a single volume. That is, the three tubes 112 are in fluid communication with each other and with the sensing unit 114. The tubes 112 are of the same dimensions in FIG. 3. Whilst three tubes are shown, it will be clear than any number of tubes may be implemented in any arrangement suitable for picking up and transmitting vibrations back to the sensing unit 114 (e.g. having multiple branches, branching at different sections, etc.)

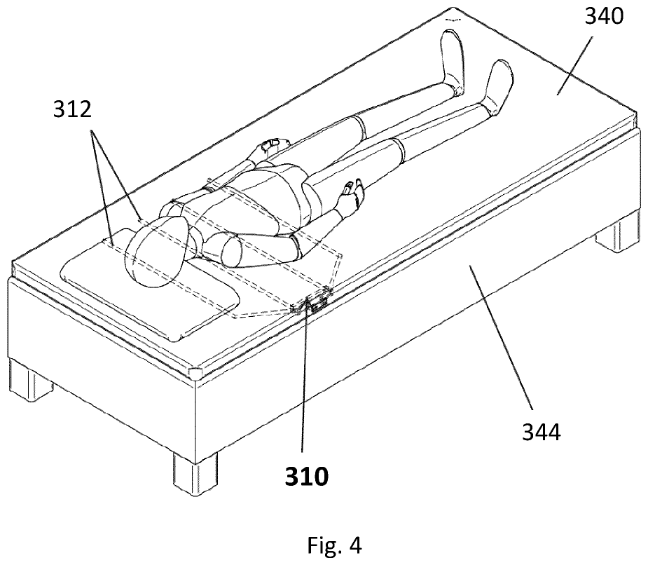

FIG. 4 shows a sleep monitoring apparatus 310 according to an embodiment embedded within a cushioning layer 340 forming part of a bed 344. The cushioning layer 340 (e.g. mattress) is placed on top of the bed 344 for a person to rest on. The cushioning layer 340 is configured to support the person at a supporting section located towards the centre of the upper contacting surface of the cushioning layer 340.

The sleep monitoring apparatus 310 is similar to that of FIG. 3; however, it comprises four tubes 312, rather than three. The tubes 310 are embedded within the cushioning layer 340, below the contact surface. The tubes 312 extend transversely across a substantial portion of the width of the cushioning layer 340 to provide a sensing area across the cushioning layer 340 over which vibrations may be detected. The tubes 310 are located towards a top end of the cushioning layer 340, so that they are located below a torso of the person once the person lies down on the bed 344 with their head towards the top end. This helps to enable the detection of heart rate and breathing rate.

Once the person comes into contact with (e.g. lies on) the sensing area, an external force 30 will be applied to the tubes 312 causing vibrations to be transmitted to the sensing unit 314.

Whilst the arrangement of FIG. 4 shows the sleep monitoring apparatus embedded within the bed in a specific arrangement, the sleep monitoring apparatus 310 may be placed anywhere within the cushioning layer to gather signals from the specific part of the bed. For instance, one or more tubes 312 may be integrated below an area for supporting the legs of a user to detect leg movement during sleep.

The vibration detected by the system may be mechanical and the sensing unit 314 may receive mechanical vibrations from the bed 344.

In one embodiment, the sensing unit further comprises a microphone. In some embodiments, the sensing unit uses both the pressure sensor and the microphone simultaneously. The sensing unit in some embodiments may use only one of a pressure sensor or microphone at any given time. In some embodiments, different signals may be captured by different sensors independently and/or simultaneously. For example, breathing and body movement can be detected by the pressure sensor(s) while heartbeat can be detected by the microphone.

The sensing unit may further comprise of other sensors that are capable of detecting vibrations, oscillations or impulses within the elastic signal collector. This also includes accelerometer(s). An example pressure sensor that can be used is a 40 kpa MPS20N0040D pressure sensor.

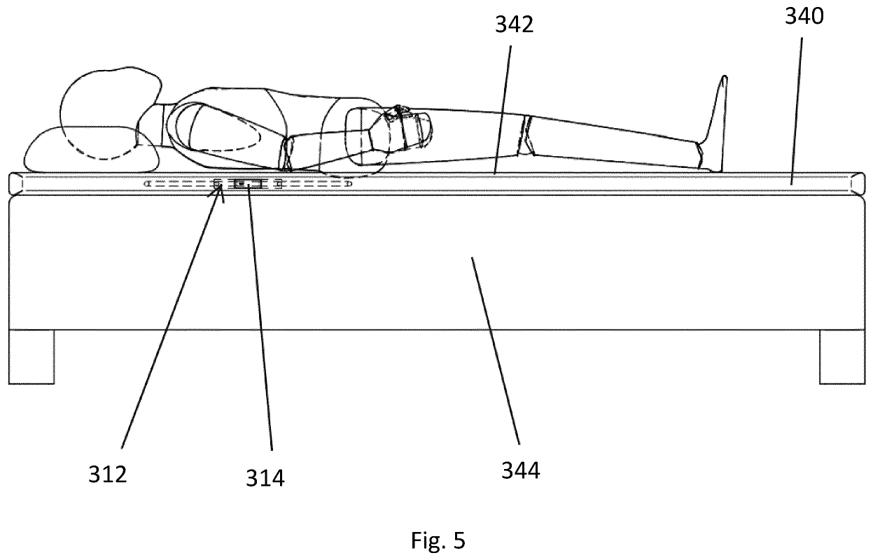

FIG. 5 shows a side view of the sleep monitoring apparatus 310 of FIG. 4. The sleep monitoring apparatus 310 may be configured to monitor the heart rate and/or breathing rate of a person lying down on the bed 344, as shown in FIG. 5. In the present embodiment, the sleep monitoring apparatus 310 is positioned within the cushioning layer 340, below in a region of the cushioning layer 340 configured to be located beneath the person's torso when the person lies on top of the cushioning layer 340. This ensures that vibrations from the heart can be transferred to the sensing unit 314 for detecting heart rate. Equally, vibrations from breathing movements (e.g. movement of the diaphragm and rib cage) are transmitted to the sensing unit for detecting breathing rate. Both heart rate and breathing rate are good indicators of sleep level (e.g. deep sleep, light sleep, REM sleep or awake).

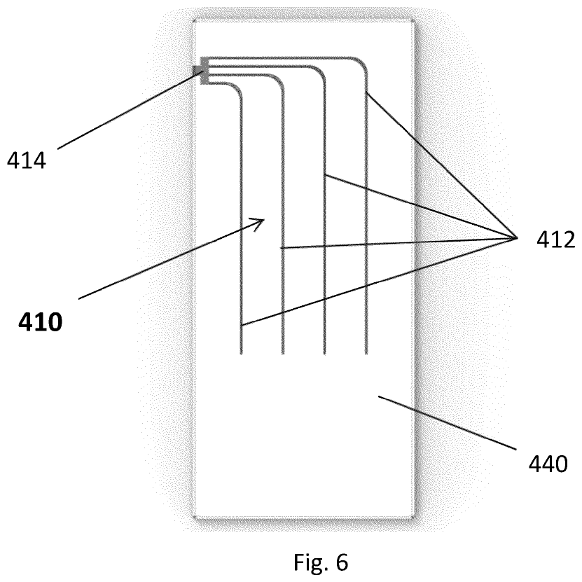

FIG. 6 shows an alternative embodiment in which tubes 412 extend longitudinally down the length of a bed. Cushioning layer 440 is in the form of a rectangular mattress. The tubes 412 comprise a bend along their length such that a larger proportion of the tube 412 may extend along the length of the cushioning layer 440, rather than the along width of the cushioning layer 440. The sensing device 414 is located towards one end of the cushioning layer 414, with the tubes 412 extending towards (and beyond) the centre of the cushioning layer 440.

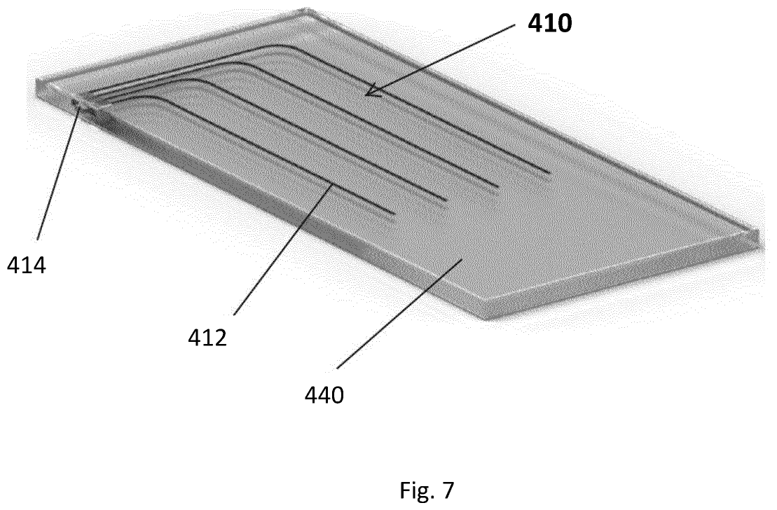

FIG. 7 shows a perspective view of the embodiment of FIG. 6. As shown here, the sensing unit 414 is embedded within a side of the cushioning layer 440.

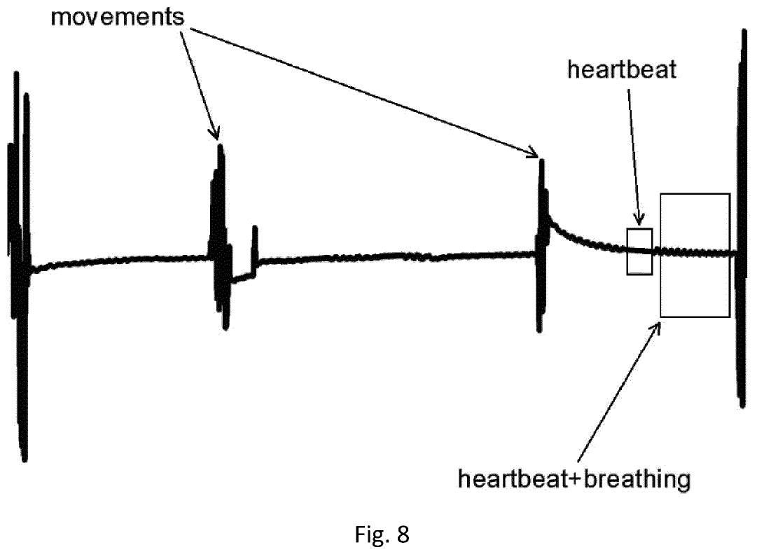

FIG. 8 shows a typical graphical representation of a pressure signal detected by a sensing unit according to an embodiment. This shows the characteristic signal patterns for body movement, heartbeat and breathing.

The length of the tube 12 influences the absolute signal amplitude received by the sensing unit 14. Different physiological properties of the person are captured and distinguished through the relative amplitudes of their signals.

FIG. 8 shows that body movements, such as turning of the body or movement of the head, legs, hands or shoulders have large amplitudes caused by their vibrations 34. The two largest peaks in beginning and end of the graphical representation relate to the person going to bed (lying down) and getting out of bed (getting up).

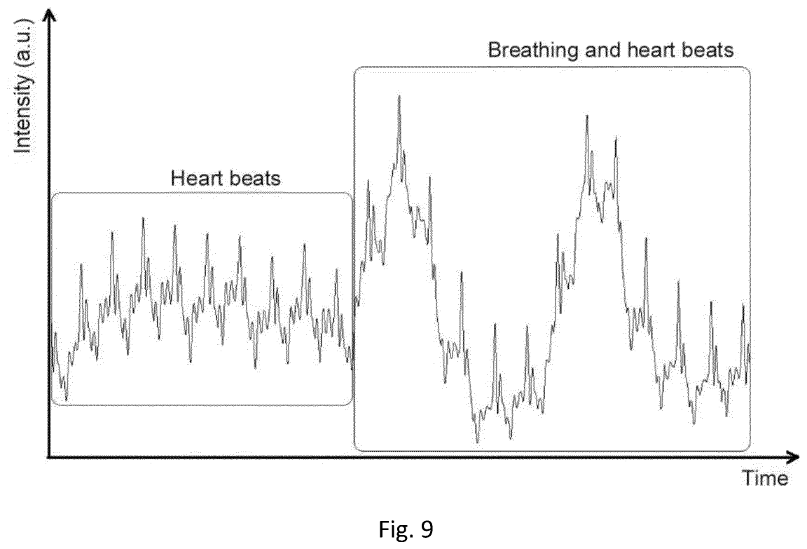

Breathing causes average amplitudes of vibrations. Heartbeat amplitudes are relatively small, but are still clearly detectable, as shown in FIG. 9.

FIG. 9 shows an intensity against time graph of the signal retrieved by the sensing unit 14 in relation to heartbeat and breathing.

Breathing is characterised by longer wavelength and higher amplitude oscillations compared to heartbeats. Heartbeat amplitude is approximately five times weaker than signals corresponding to breathing. FIG. 9 shows the intensity of the two signals being measured together. Heart rate is roughly six times higher than breathing rate.

In light of the above, the system is able to detect heartbeats, breathing cycles and body movements based on the amplitude and frequency of the detected vibrations. If vibration signal amplitude exceeds a first predefined limit then movement is detected. If vibration signal amplitude is less than the first predefined limit but above a second predefined limit (that is lower than the first), and if it falls within a first predefined range of wavelength or frequency, then breathing is detected. If the vibration signal amplitude falls below the second predefined threshold and falls within a second predefined range of wavelength of frequency, then a heartbeat is detected. The second predefined threshold range of wavelength is smaller than the first predefined range of wavelength (covers wavelengths that are lower). Conversely, if frequency is being monitored, then the second predefined threshold range of frequency is higher than the first predefined range of frequency (covers frequencies that are higher).

Based on detected movement, breathing and heartbeats, the system is configured to determine breathing rate, heart rate and rate of movement. This allows the system to characterise the level of sleep (or level of awakeness) of the user.

The combined operation of an embodiment shall now be described.

An embodiment includes a sleep monitoring system 10 comprising a cushioning layer 40 (such as a mattress or a seat), the sleep monitoring system 10 placed under a contact surface 42 and filled by fluid 20 (air in usual cases), used to collect and conduct (the tube 12) mechanical vibrations 34 inside the cushioning layer 40 to the remote (at least 5 cm or more away from signals source) sensing unit 14 for the purpose of detection of vibrations 34 caused by sleeping persons (see FIG. 5).

When external forces 30 are influencing an outer side of the sleep monitoring system 10 wall they are transmitted through the wall and transformed into mechanical vibrations 34 of fluid, packed inside the tube 12 (see FIG. 1). The sleep monitoring system 10, assembled from an elastic tube 12, is chosen in such a shape so that it transfers (or leads) vibrations from their original location to desired place(s) for the purpose of their detection by a sensing unit 14 (see FIG. 1 and FIG. 2). The elastic tube 12 is integrated into the cushioning layer 40 where it is useful as a sleep monitoring system 10 to collect vibrations 34 or oscillations caused by body movements, including breathing and heartbeat, and transport these vibrations 34 along the elastic tube 12 away for the purpose of detection.

The sleep monitoring system 10 is assembled from one or more elastic tubes 12 made of rubber or polydimethylsiloxane (PDMS) or any other elastic material that is able to transmit the signals and transform them into mechanical vibrations 34 of fluid inside the tube 12. It is important that tubes 12 restore their initial size and shape as soon the external force 30 is eliminated, to ensure continued detection. The shape of the elastic tube 12 of the sleep monitoring system 10 can be any, but is usually elongated, to enable transmission of signals over distance into a sensing unit 14, as fluid that is packed inside the elastic tube 12 (air or any other stable gas or mixture of gasses) can be applied.

Such systems (combination of an elastic tube 12 and the fluid inside) can be used as a signal collector of mechanical vibrations to lead them over the distances in the form of vibrations 34 of fluid, away from the source of the vibrations 34 (e.g. a sleeping person on the cushioning layer 40). Thus, the sleep monitoring system 10 is used to collect signals and lead them out from the cushioning layer 40, for the purpose of detection.

Detection can be implemented on the side of mattress or at least away from the body (e.g. more than 5 cm away from the body). This avoids damage to the sensing unit 14 by mechanical stress caused by the weight of the body, and avoids negatively affecting the comfort of the cushioning layer 40.

As the sleep monitoring system 10 is connected to vibration sensitive elements, when placed inside the cushioning layer 40, it can be uniquely used for detection and subsequently distinguish signals associated with body movements, body position (lying on back, side and stomach), breathing and/or heartbeat. An advantage of the sleep monitoring system 10 is that it allows the fragile electronics to be kept away from persons on the cushioning layer 40 for safety and durability as the persons can cause mechanical forces that can cause strain and even break fragile electronics. An additional advantage is that electronics are located away from the user, thereby eliminating any possible negative effects for human health caused by electromagnetic fields, caused by electric measurement systems or just to lower psychosomatics effects—for example, the worry that there would potentially be some negative health effects when sleeping near to an electronic device.

The elastic tube 12 forming the sleep monitoring system 10, is for example, an elastic pipe, which is placed between softening materials of the mattress so that motions, heartbeats and breathing of person on the cushioning layer 40 will cause mechanical force on its surface. Signal detection at the other end of the tube 12 can be achieved, for example, by a pressure sensor, microphone or some other sensor that is suitable for detection of mechanical vibrations 34. For the purposes of signal detection, more than one sensor can be connected to sleep monitoring system 10. The simultaneous use of different sensors, for example the co-use of microphone and pressure sensor, for example, would be useful to collect signals with different frequencies at the same time. This is important as different kinds of external forces 30 cause signals with different frequencies inside the collector. Thus, breathing can be detected just by pressure sensors while hearth rhythms can be detected also by microphone.

In one embodiment, the pipe forming the elastic tube 12 would be hermetic, but in alternative embodiments, the elastic tube 12 is not hermetic, for instance, it may have holes or pores inside its walls. Through those pores or holes, atmosphere (air) inside the pipe is in physical connection with atmosphere (air) outside the tube 12. The connection of inner and outer atmospheres could be useful as it enables to keep a base signal during the measurements close to corresponding value of ambient air pressure. Therefore, for example, when a person goes to bed then additional pressure inside the elastic tube 12, caused by outer force, is lost in reasonable time and further measurements can be made close to ambient air pressures. The latter would be very useful as it enables the maintenance of the pressure within the chamber within the optimum sensing range of the sensing unit 14 (this avoids excessively high pressures). Otherwise, when a person goes to bed or when he/she leaves the bed, it would cause pressure to be driven out of the sensors optimum sensing range. Thus, leaking through the wall of the tube 12 enables system to keep the sensor unit 14 operating within their optimum pressure range.

The elastic tubes 12 are to be integrated inside the cushioning layer 40 to a specific depth to get out optimal signals as well as to preserve softness of the cushioning layer 40. The exact depth of the sleep monitoring system 10 depends on the mechanical properties of sleep monitoring system 10 as well as the material of the cushioning layer 40.

The signal collector, comprising tubes 12 or a network of tubes 12, should be located close to the signal source, taking into account that the cushioning layer 40 remains between the body and elastic tubes 12 of the sleep monitoring system 10. Having said this, in some cases, for example, when using low elasticity materials, the sleep monitoring system 10 can be most effective when being in direct contact with a body, with no cushioning layer 40 between the body and collector (for instance, the signal collector might be located over the cushioning layer).

The exact form of the sleep monitoring system 10 position can vary. In an embodiment, the tubes 12 are placed perpendicular to the body in parallel jointed by T-junctions on the side of cushioning layer 40, approximately 2-3 cm from the surface of cushioning layer 40, under a layer of elastic foam within the cushioning layer 40. When integrated into a cushioning layer 40 like this, pressing the cushioning layer 40 on the top generates mechanical vibrations 34 of fluid (preferably air) inside the tubes 12. In addition to the tubes 12, a pressure sensor is utilised to detect signals (FIG. 8 and FIG. 9).

With regard to the sensing unit 14, any sensor that can detect elastic media vibrations, oscillations or impulses such a pressure sensor, microphone, accelerometer or similar can be used. In one embodiment, a 40 kpa MPS20N0040D pressure sensor is used. The length of the pipe strongly influences absolute signal amplitude. From this point of view, body (e.g. head, leg, hand, shoulder) movements cause relatively large vibration amplitudes. Breathing causes relatively average vibration amplitude. Heartbeat amplitude remains relatively low, but still clearly detectable. Corresponding signals can be seen from FIG. 8 and FIG. 9.

### Example Use of an Embodiment

A soft elastic tube (8 mm in diameter, 2 mm in wall thickness, 1 m in length), made of rubber or polydimethylsiloxane (PDMS) or some other elastic material, filled by air as gaseous fluid is integrated into the mattress under the sleeping surface. The tube is packed between soft elastic filler materials of a mattress so that approximately 2 cm thick layer of soft material will cover the tube or tubes. Air is used as the signal-conducting medium inside the tube. The tube is placed between softening materials of the mattress such that vibrations caused by a person on the mattress will force the pipe to generate mechanical vibrations that will be transferred outside the mattress by the fluid inside the pipe. A pressure sensor, used for detection of signals, is placed outside the mattress inside the far end of the pipe.

Since the particular modifications and dimensions will be apparent to a person skilled in the art, the invention is not considered limited to the said example chosen for purposes of disclosure.

### Further Embodiments

The following description below relates to further embodiments of the invention.

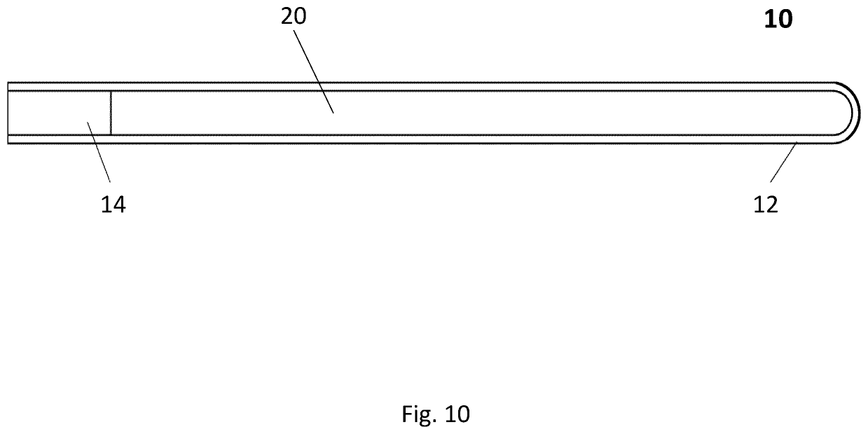

FIG. 10 shows a cross-sectional view of a sleep monitoring apparatus 10 according to an embodiment of the invention. Like numerals are used for corresponding parts relative to FIG. 1. The sleep monitoring apparatus comprises a tube 12 and a sensing unit 14. The tube 12 is fluid tight. One end of the tube 12 is sealed by the tube 12 itself whilst the other end is closed entirely by the insertion of the sensing unit 14. The tube 12 is sufficiently long to create a volume between the closed ends of the tube 12. The volume is filled with fluid 20. The fluid 20 may be any liquid or gas, but preferably air.

The tube 12 is sufficiently elastic enough such that when an external force 30 is applied to the outer surface of the elastic tube 12, it is able to return to its original shape after the external force 30 is eliminated. The Young's modulus for the elastic tube 12 may range from 0.1-100 MPa. The elastic tube 12 is preferably made out of a soft material in order for the mechanical vibrations to be captured effectively. Example materials of the tube 12 include, but are not limited to: natural rubber, synthetic rubber, silicone rubber, urethane and polydimethylsiloxane (PDMS).

The pressure sensor frequency capture range is 0-200 Hz. This allows for the effective capture of breathing rate, heart rate. The microphone's frequency capture range is also 0-200 Hz. The upper limit of 200 Hz is the optimum range to detect vibrations from heartbeat, breathing and movement. Additionally, this filters out higher frequencies that can cause interference and avoids the detection of speech, which can lead to privacy issues. Speech at the specified range (0-200 Hz) is largely incomprehensible as the speech information is generally transferred at higher frequencies.

In the present embodiment, the sensing unit 14 outputs the vibration measurements (from the microphone and from the pressure sensor) to an external device for processing. This may be via a wireless connection. The external device might be a computer, a mobile device such as a smartphone, or a central server. Nevertheless, in alternative embodiments, the processing is performed in a processor of the sensing unit 14.

The system is able to determine the user's sleep state, or level of alertness or stress, based on heart rate, breathing rate and movement rate over time. In addition to the changes in pressure, as discussed with regard to FIGS. 8 and 9, the detection of breathing rate and heartrate can be aided by detecting specific repetitive noise within breathing phase via the microphone (used to identify individual respiratory cycles or heartbeats). These noises may also be an indicator of certain diseases or sleep conditions.

Phase differences in microphone and pressure sensor signals are used to determine the body position of the user. The signals collected by the sensors depend on the relative position and orientation of the user on the cushioning layer 40. This allows detection of whether the user is on their back, on their stomach, on their left side or on their right side.

Depending on the particular position, the shape and amplitude of the resulting signal from heartbeats differs. Importantly, the signals from microphone and pressure sensor will be in phase or out of phase, depending on whether the person is lying on the back or stomach, respectively. These variations can be used to determine the position of the person lying on the mattress just from the signals collected by the tube 12, without any additional measurements.

For instance, the microphone signal can be utilised to identify individual heartbeats (or phases within a heartbeat/cardiac cycle). The pressure signal originating from heartbeat across the cardiac cycle shows the direction of movement of the heart (which is transferred into vibration in the bed). As the average location of the heart within a person is known, as well as the average motion of the heart during a cardiac cycle, the user's body position can be determined by comparing the phases of the sound and pressure signals. For example, the pressure signal measured when the person is lying on their right side has an opposite phase/direction to a signal measured when the person is on their left side. Accordingly, the sound signal can be used as a marker (trigger) in the analysis, with the reading from the pressure sensor showing the direction of movement of the right ventricle, i.e. the position of the heart in the bed.

Calibration of different positions can be carried out by the user to improve the detection of different lying positions.

Incorporation of multiple sensing units and/or multiple tubes in the mattress adds the possibility of also determining the position of person's limbs in the bed, for example, if the legs are stretched out or curled against the body. Multiple tubes enable vibration capture over a larger surface area of the mattress. These can either be connected to a single sensing unit (as discussed with reference to FIGS. 3-7, or each tube can be connected to a corresponding sensing unit. Providing a separate sensing unit for each tube allows easier separation of vibration signals from different sections of the mattress/cushioning layer.

Environmental noises can also be measured to improve the determination of the sleep state of the person. Environmental noises include, but are not limited to, noises from road traffic and electric appliances, such as air conditioner, fans and refrigerators.

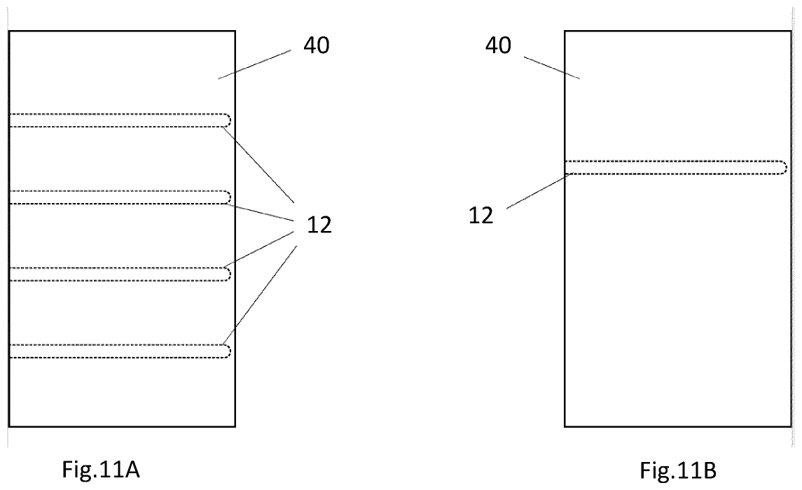

FIGS. 11A and 11B show how the sleep monitoring system may be arranged inside a mattress 40 according to an embodiment of the invention.

FIG. 11A shows a system comprising multiple independent sleep monitoring apparatuses, whilst FIG. 11B shows a system comprising a single sleep monitoring apparatus.

Each sleep monitoring apparatus 10 is positioned inside the mattress and orientated to extend along the width of the mattress, with the sensing unit located at a side of the mattress. In the present embodiments, the sleep monitoring apparatuses comprise tubes 12 that extend along the full width of the mattress. Whilst four sleep monitoring apparatuses are shown in FIG. 11A, any number of apparatuses may be utilised to provide detection at desired locations within the mattress.

Larger mattresses/cushioning layers 40 may comprise multiple sensing units and/or sleep monitoring apparatuses to capture the mechanical vibrations more effectively. In addition, multiple sets of sensing units and/or sleep monitoring apparatuses may be utilised to capture vibrations from multiple users on the same cushioning layer 40, for instance, two columns of sensing units and/or sleep monitoring apparatuses might be utilised on a double bed to capture vibrations from two users. In this case, the sensing units for the two sets could be located on oppose sides of the cushioning layer, with the tubes extending towards the centre, on opposite halves of the cushioning layer 40.

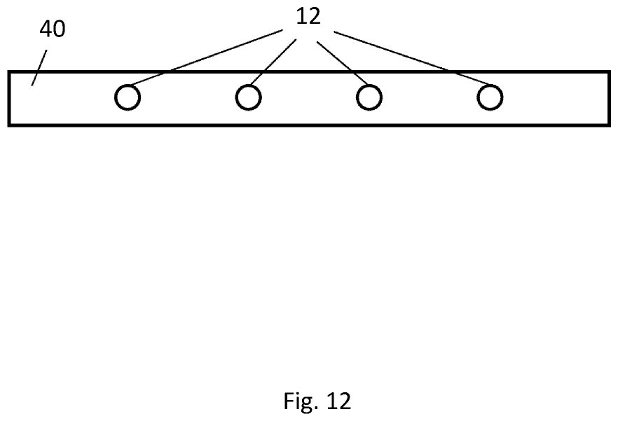

FIG. 12 shows a side view of the sleep monitoring system 10 of FIG. 11A. The tubes 12 arranged in regular intervals along the length of the cushioning layer 10. However, in some embodiments, the tubes 12 may be arranged such a way that the collection of vibrations 34 may be focused on certain contact areas 42. For example, more tubes 12 may be positioned at the top part of the mattress to collect vibrations 34 from the person's head and torso.

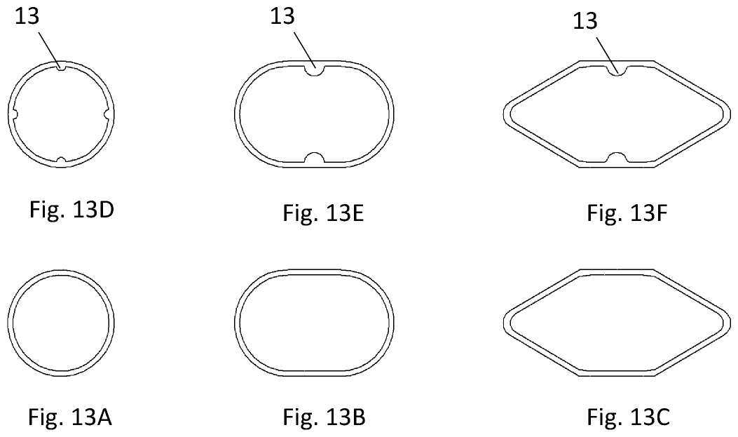

FIG. 13A-13F show examples of different cross sections for the tube 12 according to various embodiments. The shape of the tube is important for transmitting vibrations 34 through pressure changes in the fluid 20. For example, a regular cross-section along the length of the tube helps to avoid reflections and/or noise when transmitting vibrations 34 through pressure changes in the fluid 20.

FIG. 13A shows a tube having a circular cross-section. FIG. 13B shows a tube having a cross-section having a central elongate section with two rounded ends. A similar embodiment might have an elliptical cross-section. FIG. 13C shows a tube having a cross-section having a central elongate section and two pointed ends.

FIGS. 13D-F show tubes having similar cross-sections to those of FIGS. 13A-C; however, inner protrusions are provided to reduce the risk of the tubes becoming stuck shut.

Due to the strength of the potential external forces 30 could be exerted on the tubes 12, the tubes 12 may be forced completely closed at certain points along the length of the tube. In this case, it is possible for the tube to stick shut when collapsed. This problem is solved by providing inner protrusions 13 inside the tube 12 to reduce the surface area of the inner wall that touches when the tube 12 is forced shut.

FIG. 13D shows a circular tube having four protrusions 13, spaced evenly around the circumference of the tube. FIG. 13E shows a tube similar to that of FIG. 13B but having two protrusions on opposite sides of the central elongate section. Equally, FIG. 13F shows a tube similar to that of FIG. 13C but having two protrusions on opposite sides of the central elongate section.

Each protrusion is a ridge that extends along the length of the tube. This provides a constant cross-section across the length of the tube, thereby reducing backward reflections within the tube.

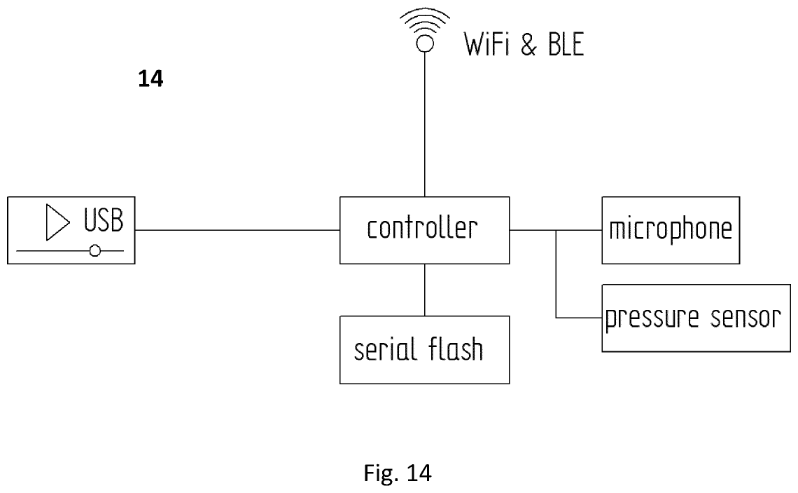

FIG. 14 shows a sensing unit according to an embodiment. The sensing unit 14 comprises a pressure sensor, a microphone, a controller, memory in the form of serial flash, a USB port and an input/output module in the form of Wi-Fi and Bluetooth modules.

The microphone is configured to detect vibrations in the form of sound within the tube. The pressure sensor is configured to detect pressure within the tube. The microphone and pressure sensor are connected to the controller and report their measurements to the controller. The controller is configured to store measurements in the memory (the serial flash) and to output measurements via the output module (e.g. the Wi-Fi or Bluetooth modules) or via the USB module. The system is powered via a battery or via a mains power supply, e.g. via the USB port.

Whilst the embodiment of FIG. 14 includes a serial flash, any form of memory might be utilised. Equally, whilst USB, Wi-Fi and Bluetooth modules, any appropriate form of output port output module might be utilised.

The memory stores computer executable instructions that, when executed, cause the controller to perform the functions described herein. Whilst the present embodiment outputs vibration measurements to an external system for analysis, alternative embodiments perform analysis of the measurements via the controller of the sensing unit.

The sensing unit 14 may a cross-section that corresponds to the internal cross-section of the tube 12 to allow the unit 14 to easily fit within the tube 12. Having said this, the sensing unit 14 is not required to have a cross-section that corresponds precisely to the internal cross-section of the tube 12 to work the invention, provided that at least a coupling section of the sensing unit 14 is configured to be inserted within the tube 12, for instance, by at least fitting within the tube 12 (having cross-section dimensions, e.g. width and/or height that are less than the inner diameter of the tube), if not having a corresponding cross-section to the tube 12. For instance, the coupling section might have a square or rectangular cross-section, with the tube 12 being sealed around the coupling section, e.g. via glue.

To assemble the system, the sensing unit 14 is first inserted into one end of the tube 12 that is closed on the other end. The sensing unit 14 is inserted deep enough to leave one end (in which the USB port is located) flush with the open end of the tube.

The end of the tube with the inserted sensing unit 14 is thereafter hermetically sealed (e.g. by heat shrinking, using non-conducting glue, or both). The tube can be filled with various gasses before sealing. Alternatively, the sensing unit 14 may be held within the tube 14 via an interference fit; although this might result in air escaping the tube 14 during use.

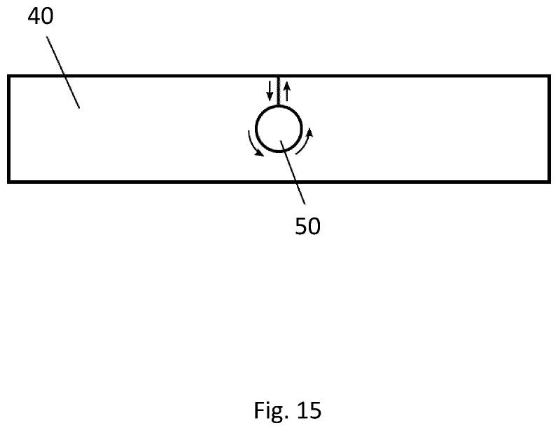

FIG. 15 shows a manufacturing process for the integration of the sleep monitoring apparatus 10 into a cushioning layer 40. The manufacturing process includes a cutting tool specifically designed for carving channels 50 into a cushioning layer 40.

Cushioning layers, such as mattresses, can be difficult to cut, due to their compressibility and resilience. Accordingly, a customized cutting tool is provided that makes the cutting of such cushioning layers easier.

The cutting tool comprises a diamond-coated wire of, for instance, steel. The wire is a formed into a loop and is driven via a motor (for instance, via a set of pulleys). By coating the wire with diamond (or with another suitably hard substance), a particularly hard and sharp edge can be provided for cutting.

The cutting tool functions in a similar manner to a band saw, with an exposed region having the wire running through it to provide an area for cutting the cushioning layer 40.

FIG. 15 shows a view down the length of a cushioning layer 40 (e.g. a mattress). In this view, the wire of the cutting tool runs perpendicular to the view (out of the page). A channel may be cut down the length of the cushioning layer 40 by urging the cushioning layer against the wire and moving it along the path shown in FIG. 15. The wire enters a top layer of the cushioning layer, forms a straight entry/exit path, then forms a loop/circle for cutting the channel, then exits through the entry/exit path.

In use, the cutting tool may be kept stationary (with the wire being driven by the motor) and the cushioning layer 40 may be urged against the wire to cut the desired shape (the shape of the channel 50 for receiving the tube). The cushioning layer 40 can be moved by hand or by a mechanical stage (e.g. computer controlled). Alternatively, the cushioning layer 40 may be kept stationary with the cutting tool being moved along a desired path to cut the channel 50.

The cushioning layer 40 can be compressed to enable cutting of mattress foams thicker than the length of the exposed cutting wire. In one embodiment, the channel 50 for the tube 12 is to be cut such that the distance between the top of the channel 50 and top of the cushioning element 40 is sufficient to avoid the tube 14 impacting the comfort of the cushioning layer, but shallow enough to ensure that vibrations are still picked up by the tube (e.g. at least 5 cm from the surface of the cushioning layer).

Finally, the tube 12 is inserted into the channel 50 in the cushioning element 40. The tube 12 can be freestanding (e.g. secured via an interference fit) in the channel 50 or alternatively, the channel 50 or tube 12 walls can be covered with glue prior to inserting the tube, in order to fix its position to avoid unwanted shifting and collapse of the tube 12 during the use of the cushioning layer 40.

The entry/exit point used for cutting the channel 50 can be left open (for instance, with the tube 12 being secured via glue within the channel 50 or an interference fit within the channel 50, or with the cushioning layer 40 being otherwise sealed, such as through containment within an outer cover). Alternatively, the entry/exit path can be sealed shut, e.g. via glue.

### Analysis of Raw Data and Data Streaming

The controller of the sensing unit is able to communicate directly with an external computing system, such as a mobile device, computer or server, through wireless communication, e.g. Bluetooth or Wi-Fi communication. In one embodiment, the sensing unit communicates with a user's mobile device (e.g. a smartphone). If the external device is not in range, or connection is lost through another means, then the data is stored and then transmitted to the external device when connection is restored.

The signal may be processed on the sensing unit 14, e.g. to remove noise and to compress the signal, and the processed signal can be sent to the external device for tasks that require additional computing power. In some alternative embodiments, a raw, unprocessed feed of data is sent to the external device for more precise analysis for the sensed data.

It is also possible to detect heart and breathing related diseases from the signals picked up by the sensing unit 14. The detection of these conditions may require more computing power than provided by the on-chip microprocessor in the sensing unit 14, and comparisons with external databases might be necessary. For example, spectral analysis may be needed, which requires a Fourier transform of the raw sensor signal. Additionally, as these disease conditions manifest themselves as intricate and sometimes small deviations in sound spectra compared to normal healthy state, the raw data used for such analysis should have minimal noise.

In order to carry out such analysis, the signal from the sensing unit 14 is monitored by the software implemented on the controller overnight. To avoid exporting or storing large quantities of data, the system might monitor the raw data and only export or store a predetermined period (e.g. a short, burst) of raw data if certain conditions are met (e.g. when a good quality of signal is detected, and/or when a specific sleep level is detected).

The duration of the burst may be up to five minutes. Analysis of the raw data can take place in the external device or the data can be sent to a remote server to access additional computing power. Due to the high data rate of the raw data, it should be ensured that optimal conditions are present for collecting high quality data, so as not to waste energy and storage space for data that might not be relevant for further analysis.

Four parameters are monitored to determine the start of a data stream session and when all four conditions pass threshold values, a data stream is initiated. The thresholds may vary, depending on whether heart or breathing sounds are to be analysed.

The four parameters to be met for sending a data stream to a phone or remote server for heart/breathing condition analysis are:
(1) sleep state is deep sleep;
(2) there have been no movements in a predetermined period of time (e.g. the last five minutes);
(3) moving average root-mean-square (RMS) value of the signal has not changed by more than a predefined amount over a predetermined period of time (e.g. 20% in the last five minutes); and
(4) heart/breathing rate is detected.

The times and percentages in conditions (2) and (3) can vary (e.g. 1-20 minutes and 5-50%). Once these conditions are satisfied, the sensing unit 14 shall record and export the signal (e.g. over a predefined period or burst) for further analysis, for instance, exporting to memory and/or to an external device.

### Heart Rate Measurement

To detect heart rate, the frequency range of 10-30 Hz is filtered out from the raw heartbeat signal (e.g. via a band-pass filter). That is, frequencies outside of the range of 10-30 Hz are suppressed. The resulting heartbeat signal (over the 10-30 Hz range) is smoothed with a moving average method. Two passes of smoothing may be performed for improved performance. Local maxima are detected from the resulting signal. Every heartbeat gives two peaks (local maxima), one of them is primary and has a higher amplitude, and the other one has lower amplitude. The lower secondary peaks are removed and primary peaks are used to determine heart rate. That is, each primary peak represents a corresponding heartbeat. The time between primary peaks can be determined and utilised to determine frequency (e.g. by dividing the number of primary peaks with the time over which the primary peaks are measured). A moving window may be utilised to average out the heart rate over the window.

### Breathing Rate Measurement

Breathing rate measurement is performed without frequency range cutting. The raw signal is smoothed with the above-mentioned moving average method (e.g. using two passes). Peaks (local maxima) are identified for calculating breathing rate. For determining peaks, peaks that have an amplitude below a certain threshold amplitude and/or a frequency above a certain threshold frequency are disregarded as noise. This is achieved by determining the relative amplitudes and time separation between peaks so that peaks relating to small, high frequency fluctuations (i.e. that are unlikely to relate to breathing) are filtered out. The peaks within the filtered signal are then utilised to determine breathing rate. That is, each peak represents a corresponding breath (a single cycle of breathing). The time between peaks can be determined and utilised to determine frequency (e.g. by dividing the number of peaks with the time over which the peaks are measured). A moving window may be utilised to average out the breathing rate over the window.

### Sleep Level Classification

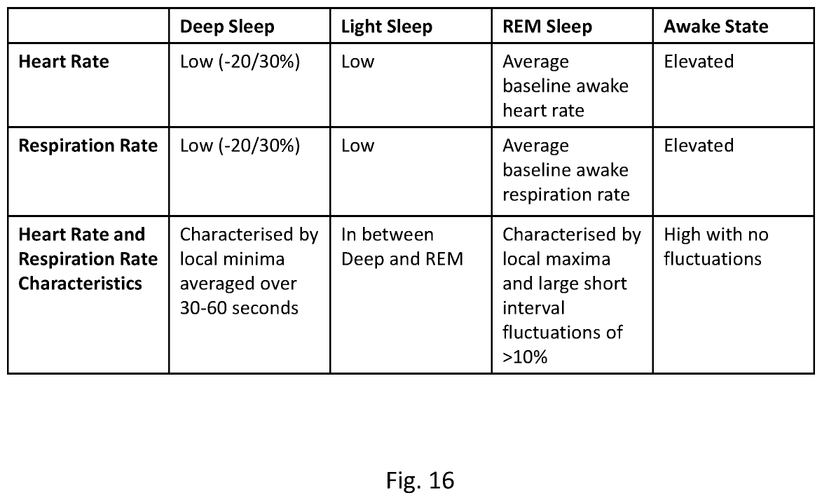

FIG. 16 shows a table detailing a method for determining a sleep level of the user according to an embodiment. A person goes through several stages of sleep throughout the night, broadly categorized as light sleep, deep sleep and rapid eye movement (REM) sleep. The sleep stages are cyclic and vary from light sleep to deep sleep and back to light sleep. At the end of each such cycle, REM sleep occurs. Each stage of sleep is associated with changes in heart rate and respiration rate. Periods of wakefulness can also occur. Compared to the awake state, light and deep sleep are characterized by lowered heart rate and respiration, with deep sleep having the lowest values (approximately 20-30% lower than awake).

The baseline awake heart and respiration rates for each individual user can be determined at the time the person goes to bed, before falling asleep. Long-term average awake heart and breathing rates can be recorded for using as a parameter for determining sleep states. During REM sleep, the heart and respiration rates increase to levels comparable to awake state, accompanied by above average variations in both rates. Following the decrease and increase of heart rate and respiration, and taking into account larger variation of both during REM sleep stage, sleep stages can be assigned. Deep sleep stages correspond to the local minima in the heart and respiration rate curves, averaged over 30-60 second intervals. REM sleep similarly corresponds to local maxima accompanied by large short interval fluctuations of >10%. Light sleep falls between deep and REM sleep in terms of heartrate and breathing rate. Being awake is characterized by elevated heart rate and respiration compared to light and deep sleep, but without fluctuations characteristic to REM sleep.

From this data and data obtained by the sensing unit 14, software on the external device, or on the sensing unit, is able to determine a curve of sleeps states (e.g. deep sleep, light sleep, REM sleep, awake).

In light of the above, a first set of thresholds may be set for the heart rate and/or breathing rate. If the heartrate and/or breathing rate are above this first threshold, and the variability in heartrate and/or breathing rate is below a corresponding threshold variability, then the system might classify the user as awake. If the heartrate and/or breathing rate are above the first threshold, and the variability in heartrate and/or breathing rate is above the corresponding threshold variability, then the system might classify the user as in REM sleep. If the heartrate and/or breathing rate are below the first threshold, but above a second threshold (that is less than the first threshold), then the system might classify the user as in light sleep. If the heartrate and/or breathing rate are below the second threshold then the system might classify the user as in deep sleep.

The system may classify the user as falling within one of the above classes if one or both of the heartrate and breathing rate fall within the corresponding limits described above.

Method for Determining when to Wake Sleeping Person

The systems described here might be applied to determine the optimum time to wake a sleeping person (via an alarm) based on the sleep state of the person. Waking up during deep or REM sleep can cause a person to feel groggy and disoriented. Accordingly, there are advantages to monitoring sleep state to ensure that the user is woken at the appropriate time within the sleep cycle in order to ensure that they wake up feeling well rested.

A user can set a preferred time window for wake up time, specifically the earliest and latest time an alarm should sound, which can also be referred to as the start and end time, respectively. The predefined end time ensures that the user does not over sleep and miss any prearranged appointments. The predefined start time ensures that the amount of sleep is maximised whilst also allowing a period of time for selecting the most appropriate time to wake the person based on sleep state.

The sleeping person's stages of sleep are monitored throughout the night. The preferred time to wake up a person is once the person has returned to light sleep after completion of REM sleep. An incomplete and interrupted REM stage can result in a sensation of sleepiness for an extended period after wakeup.

Alternatively, if the above sleep state transition (REM to light sleep)_is not observed, then it is still preferable to wake the user during light sleep than during deep or REM sleep, as this would still provide reduced drowsiness in the user.

Accordingly, if the user is in, or enters, REM sleep during the pre-defined wake up time interval, but no later than 15 minutes before the end time, the alarm will sound as soon as the user completes the REM stage and enters into light sleep stage or at the pre-defined latest alarm time, whichever comes first.

If the user is in light sleep stage within a predefined period (e.g. 15 minutes) before the pre-defined end time (has not yet been in REM sleep during the alarm interval), the alarm will sound 15 minutes before the latest time to avoid entering REM stage for a short period of time.

If the user is in deep sleep 15 minutes before the pre-set end time (has not yet been in REM sleep during the alarm interval), the alarm will sound as soon as the user enters light sleep stage or at the pre-set latest alarm time, whichever comes first.

The 15 minute time-point is indicative and can be varied for example in the range of 5-30 minutes. The difference between earliest and latest time that a user can set should be equal or greater than this time-point.

A snooze function can be added to the alarm, which when activated after the alarm sounds, will stop the alarm and sound it again after certain pre-set time (e.g. 1-15 minutes).

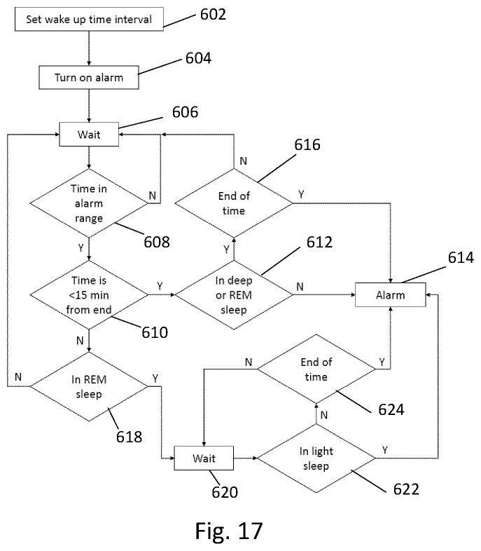

FIG. 17 shows a flow chart for a method of determining when to activate an alarm based on determined sleep levels according to an embodiment.

This method may be implemented within a controller within the sensing unit or may be implemented within an external device, such as a user's smartphone, that either receives vibration measurements and determines the sleep level itself, or receives reports of sleep level from another device (that bases these determinations of vibration measurements).

A first step 602 comprises the setting of a wake up time interval. This is the interval (an alarm period) over which the person wishes to be woken up by the sleep monitoring system 10. An end time is set up to define the latest time the alarm can be issued. A start time is set up to define the earliest time the alarm can be issued. The start time and end time are received via inputs from the user (e.g. via an input device such as a key board or touch screen, or via a connection with a further device, such as via wireless communication).

The system then receives an input to activate the alarm functionality 604. This instructs the system to monitor sleep activity and time, relative to the alarm period, and to issue an alarm to wake the user once the required criteria have been met.

The system then waits 606, monitoring the current time, until the start of the alarm period is reached (i.e. until the current time is equal to or later than the start time).

The system determines whether the current time is within the alarm period 608. If not then the system returns to step 606 to continue to wait. If the current time is within the alarm period 608 then the system then determines whether to issue an alarm to wake the user up.

The system then determines if the current time is less than a predetermined period (e.g. 15 minutes) from the end time 610. The predetermined period is shorter than the alarm period, so this step is only executed if the current time is in the alarm period. This step checks if the wake up time interval is nearing its end, in which case the system issues an alarm whenever the user is outside of deep or REM sleep, instead of waiting for a change from REM to light (as described below) to avoid the user entering REM sleep and not leaving REM sleep before the end time. 15 minutes is an arbitrary time period, although it is chosen as indicative of a time appropriate for avoid REM sleep from continuing on to the end time. Other lengths of predetermined periods may be used to achieve this function.

If the current time is within the predetermined period, then the system of determines if the person is in deep sleep or REM sleep 612. If not (e.g. the user is awake or in light sleep), then the system issues an alarm 614. If the user is in deep or REM sleep, then the system determines if the end time has been reached 616. If not, then the system loops back to step 606 and the method repeats. If the end time has been reached, then the alarm is issued 614.

If, during step 610, it is determined that the time is not within the predetermined period, then the system determines whether the user is in REM sleep 618. If not, then the system loops back to step 606. If the user is in REM sleep at this point, then the system waits 620, to see if the user will leave REM sleep before the end time.

The system checks whether the user has entered light sleep 622. If so, then the alarm is issued 614. If not, then the system determines whether the end time is reached 624. If so, then the alarm is issued 614. If not, then the system loops back to step 620 to continue to wait to see if the user will enter light sleep.

When an alarm is issued, it may either be an alert that is physically transmitted by the system (e.g. a sound and/or light) or it may be an electronic alert that is issued to another device to wake up the user (e.g. a wireless transmission to an alarm to issue a sound and/or light to wake up the user).

The ordering of the above steps are not necessarily the only order in which embodiments may be implemented. Some steps may be interchangeable and still achieve the same technical effect.

In light of the above, embodiments of the present invention provide a sleep monitoring system that is configured to detect mechanical vibrations for determining one or more physiological parameters (such as heart rate or respiration rate) and/or one or more physiological states (such as sleep level) of a person situated on a cushioning layer. This is achieved by providing a fluid-filled elastic tube connected to a sensing unit that can detect vibrations in the fluid caused by an external force on the elastic tube. A controller (either within the sensing unit or in an external device) is then able to determine the physiological state of the person depending on the user's heart rate, respiration rate, body position and/or movement based on the vibration sensed in the elastic tube. By providing a signal collector in the form of an elastic tube, vibrations are transmitted away from the body of the user, thereby allowing the sensing unit to be located away from the body, protecting the sensing unit from mechanical stress and avoiding any negative impact on the comfort of the cushioning layer that might be provided through the embedding of a hard sensing unit close to the user's body within the cushioning layer.

Further embodiments comprise an alarm system for waking up a sleeping person depending on the physiological state of the person, wherein the physiological state of the person can include stages of sleep. Stages of sleep include: deep sleep, light sleep, REM sleep and awake; which are also determined based on the vibrations of the fluid. By making use of the sleep state of the person, the alarm may be timed to wake the user at the most optimum point within the sleep cycle to avoid drowsiness.

It is noted that whilst most of the embodiments discussed above have been related to sleep monitoring, the above mentioned data and sleep monitoring apparatus 10 may be used for different types of physiological monitoring and utilised for different users, including, but not limited to:
Sleep monitoring for personal use (health and lifestyle)
Sleep monitoring for controlling external equipment—turning off equipment that might disturb sleep via sounds or light (e.g. fans, air conditioning, etc.) during light sleep stage
Monitoring of elderly (personal and for caregivers)
Monitoring of meditation (e.g. mindfulness)
Monitoring of diseases and health problems (e.g. epilepsy, insomnia, snoring, sleep apnoea, heart attack, stroke)

Whilst the embodiments described herein have been described with reference to implementation within a bed or mattress, the methods described herein are applicable to other forms of cushioned furniture, or cushioned products. For instance, embodiments might be integrated within the backrest of a chair to monitor heart rate, breathing rate, movement, etc. This could be useful for determining stress levels and levels of awakeness. Equally, the embodiments might be implemented into the padding of a backpack to monitor heart rate and breathing rate. More generally, the embodiments can be implemented in any cushioning layer that is configured to be urged against the body of a user (or have a user's body urged against it), and in particular, against the torso of a user (for improved detection of respiration and heartrate).

### Signal Processing

Methods for processing pressure 180 and audio signals 182 shall now be described. The processing may be performed either in the sleep monitoring apparatus 10, a mobile device or other computing device connected to the sleep monitoring apparatus 10, or in an external server to which the sleep monitoring apparatus 10 reports.

As discussed above, the sleep monitoring apparatus 10 detects pressure 180 and audio signals 182 through a signal collector. Transmission of the acoustic signal 182 and co-transmission of acoustic 182 and pressure signal 180 with an elastic tube 12 as the signal collector is delivered by a smooth inner surface present in the elastic tube 12. The smooth surfaced signal collector tube 12 enables reflection of sound waves on the inner surface of the tube with minimal signal losses, by improving wave propagation. The elasticity of the elastic tubes 12 may be modified to provide optimal comfort for the user.

Data analysis and determination mentioned herein may be made through conventional deterministic computation methods or more probabilistic, stochastic methods such as machine learning algorithms.

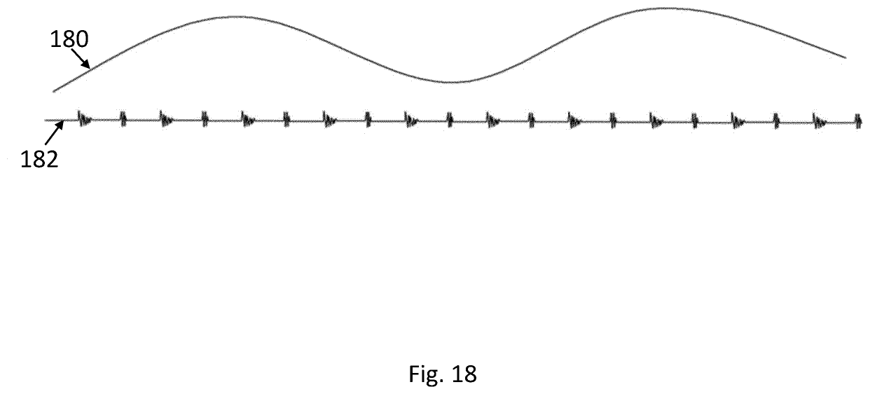

FIG. 18 shows a pressure signal 180 representing pressure within a cushioning layer 40 supporting at least a portion of a user and an acoustic signal 182 representing acoustic vibrations within the cushioning layer 40. The pressure signal 180 and the acoustic signal 182 are received by one or more processors that are present in a system for use in monitoring one or more physiological states of a user, such as that described with reference to FIGS. 1-17.

The system for use in monitoring one or more physiological states of the user is carried out by the co-use of multiple sensors. The pressure signal 180 representing pressure within the cushioning layer 40 may be provided by sensors, which are capable of detecting pressure these include, but are not limited to, electrical capacity sensors, piezoelectric sensors and accelerometers. The acoustic signal 182 representing acoustic vibrations within the cushioning layer may be provided sensors, which are capable of detection acoustic vibrations these include, but are not limited to microphones.

Both the pressure signal 180 and acoustic signal 182 contain information for various physiological parameters of the user, such as respiration, breathing, breathing rate, sounds synchronous with breathing, heartbeat, heart rate, sounds synchronous with heart rate; however, at different levels of amplitude depending on whether it is detected from the pressure signal 180 or acoustic signal 182.

Moreover, environmental noise can be detected by the system for use in monitoring one or more physiological states of the user. For example, environmental noise level can be determined by measurements by the sensors when the user is absent. The environmental noise can be subtracted or filtered from the information for physiological parameters of the user to improve the results. Environmental noise can also be taken into account to provide feedback on poor sleep quality or to control disturbing devices, for instance, by turning off devices that are causing excessive noise levels when the user is sleeping and/or within a specific sleep level (e.g. REM sleep). Other noises not related to the environment of the system can also be taken into account to provide feedback on poor sleep quality.

The pressure signal 180 represents pressure oscillations at a lower frequency than the acoustic vibrations. Accordingly, the one or more processors receive pressure signals 180 of lower frequency than the acoustic signals 182. This is implemented by the pressure signal 180 and acoustic signal 182 having different frequency capture ranges.

Alternatively, filters may be applied to the pressure 180 and acoustic signals 182 to filter the signals into different frequency ranges. The co-use of different frequency capture ranges enables the one or more processors to obtain information regarding different physiological parameters of the user from the pressure signal 180 and the acoustic signal 182 to determine, based on the pressure signal 180 and acoustic signal 182, one or more physiological states of the user in an efficient and effective manner.

The different frequency capture ranges of the pressure signal 180 and acoustic signal 182 enables the one or more processors to receive signals indicative of a physiological parameter that is desired to be monitored at a frequency band in which the signals indicative of a physiological parameter that is desired to be monitored have a higher amplitude and/or signal to noise ratio.

For example, information on the user's breathing and heartbeat is required to determine presence of a variety of abnormal physiological parameters. For instance, the system can detect the presence of an abnormal sound synchronous with breathing of the user, the co-use of the pressure signal 180 and acoustic signal 182 allow the efficient and effective determination of the physiological parameter through their respective frequency capture ranges.

Abnormal sounds synchronous with breathing include, but are not limited to, snoring, wheezing, crackling, stridor, rhonchi or pleural rubs. These abnormal sounds are an indication of various disease conditions and abnormal physiological states of the user, examples include obstruction of airways, asthma, pneumonia, bronchitis, inflammation, air or fluid in or around the lungs, chronic obstructive pulmonary disease, bronchiectasis and particularly any other obstructive pulmonary diseases that cause abnormal sounds in the lungs.

Breathing by the user will cause significant changes in the pressure of the cushioning layer 40 due to the expansion and contraction of the user's lungs. Breathing by the user, whilst detectable by the acoustic vibrations in the cushioning layer 40, may not have sufficient amplitude to stand out from potential noise in the frequency capture range at which the acoustic sensor operates. Therefore, information on breathing of the user is obtained from the pressure signal 180.

This may be achieved by extracting a respiration signal from the pressure signal 180, e.g. by applying one or more filters that pass the respiration signal but blocks other pressure signals. Having said this filtering may not be needed dependent on the difference in active frequencies of the pressure and acoustic sensors. Equally, if the user is showing negligible movement on the cushioning layer 40, for example by being in a deep sleep state, the pressure signal 180 may purely reflect the respiration signal through the raw signal itself directly from the pressure sensor. The respiration rate is determined by measuring the frequency of the respiration signal. The respiration signal is a component of the pressure signal related to respiration.

In addition, the heart rate is monitored through the extraction of a heart (or cardiac) signal from the acoustic signal 182. Again, this might be through application of one or more filters that pass the heart signal but block other acoustic signals (such as external noise). Having said this filtering may not be needed dependent on the difference in active frequencies of the pressure and acoustic sensors. Equally, if there is negligible noise from the environment surrounding the sleep monitoring apparatus 10 and the user is in a deep sleep state resulting in negligible noise from movement of the user, the acoustic signal 182 may purely reflect the heart (or cardiac) signal through the raw signal itself directly from the acoustic sensor. The heart rate is determined by measuring the frequency of beats within the heart signal. The cardiac signal is a component of the acoustic signal related to cardiac activity of the user.

For example, the heart rate may be determined by averaging and filtering the received acoustic signal 182, detecting maxima(s) in the averaged and filtered signal, identifying start and end points of the maxima detected signal (if any) and applying an amplitude threshold filter, such as an expander or compressor, to the acoustic signal to remove any noise that reduces the quality of the heart signal.

Alternatively, or in addition to the application of one or more filters to the pressure signal 180 and/or the acoustic signal 182, the respiration signal and heart signal may be extracted using machine learning algorithms to differentiate the respective signals from their source signals.

For example, the heart rate may be determined by a machine learning classification or clustering algorithm that is configured to identify heart signals with heart rates (e.g. within the range of 50-90 beats per minute).

Heartbeat of the user can be detected using two methods recognized as phonocardiography (PCG) and ballistocardiography (BCG). BCG can be used by detecting pressure changes in the elastic tube 12 of the sleep monitoring apparatus 10 as described above. PCG uses low frequency operation of microphones to detect and monitor heartbeat of the user.

Abnormal sounds of the user will cause significant changes in the acoustic vibration of the cushioning layer due to distinctive heart or breathing sounds of the user. Abnormal sounds of the user, whilst detectable by the pressure in the cushioning layer, may not have sufficient amplitude to stand out from potential noise in the frequency capture range at which the pressure sensor operates. Therefore, information on abnormal sounds the user is obtained from the acoustic signal.

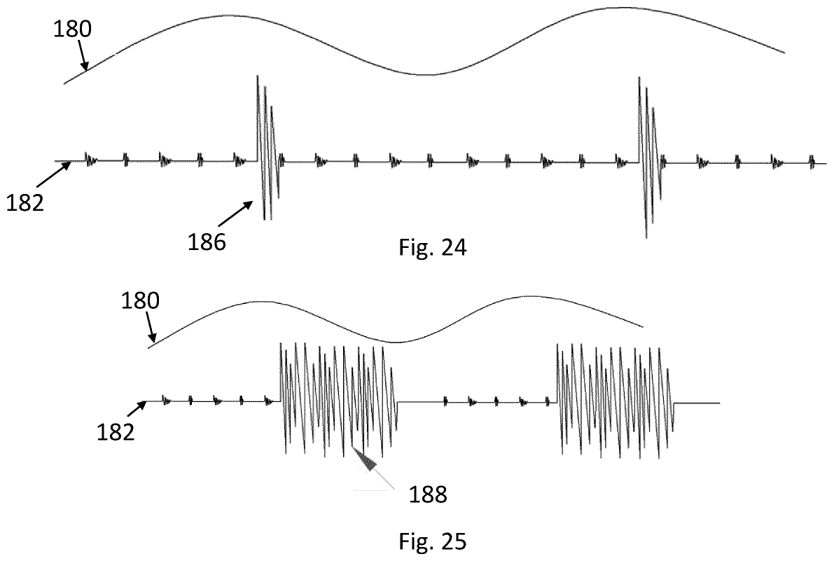

The one or more processors will determine the presence of an abnormal sound synchronous with breathing of the user through the co-use of the pressure signal 180 and acoustic signal 182. A detailed description of the determination of the presence of an abnormal sound synchronous with breathing of the user will be discussed in further detail in relation to FIG. 24.

It should be noted that the co-use of pressure signals 180 and acoustic signals 182 can be implemented to determine one or more physiological states in parallel. Furthermore, different frequency capture ranges can be implemented in parallel; this is particularly desirable if monitoring of multiple of physiological states are required.

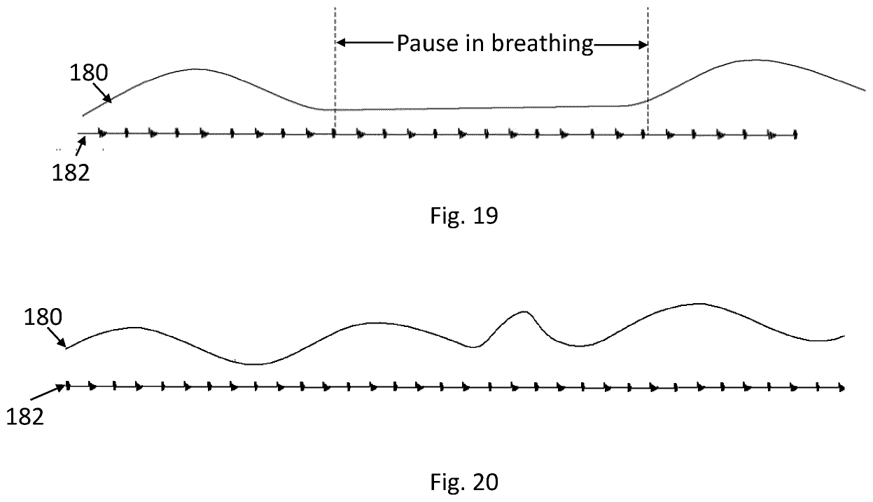

FIG. 19 shows a graphical representation of pressure 180 and acoustic signals 182 showing an interruption in breathing of the user. An interruption in breathing includes stoppages or shallowness in breathing. An interruption in breathing is determined in response to a detection of an absence of change in the respiration signal of the user for a predetermined duration.

An interruption in breathing of the user is only determined by the one or more processors if a heart signal remains present in the acoustic signal 182 received by the one or more processors. The absence of a heart signal could indicate that another physiological state should be determined (or that the user is not present on the cushioning layer 40).

Changes in heart rate are possible during the interruption in breathing. For example, the heart rate could increase to compensate for lowered oxygen levels in the blood.

An interruption in breathing of the user may also be indicative of sleep apnea of the user. Sleep apnea can be detected as a specific case of interruption in breathing by detecting an interruption in breathing when the user is sleeping (by monitoring level of consciousness or sleep level of the user). Snoring sounds and respiratory noises synchronous with breathing are also a side effect of sleep apnea, which can be determined in parallel with the determination of an interruption in breathing of the user.

FIG. 20 shows a graphical representation of pressure 180 and acoustic signals 182 showing an irregularity in breathing of the user. An irregularity in breathing includes changes in frequency of breathing. An irregularity in breathing is determined in response to a detection of irregular changes in frequency in the respiration signal of the user detected in the pressure signal 180. An irregularity in breathing can also be determined in response to a detection of variation in frequency in the respiration signal of the user detection in the pressure signal above a frequency variation threshold.

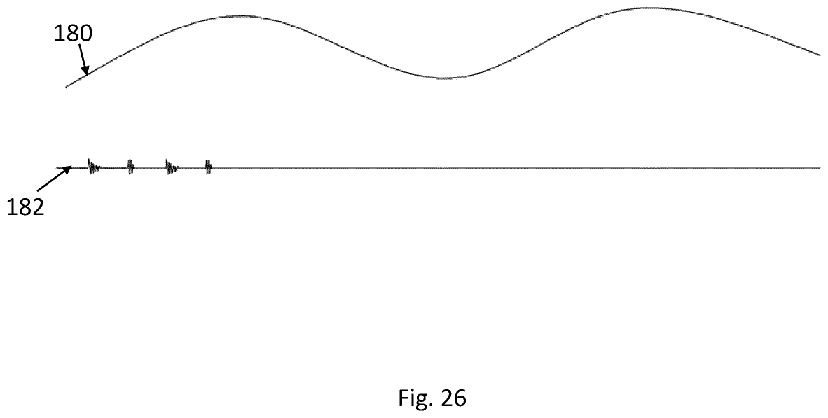

An irregularity in breathing of the user is only determined by the one or more processors if a heart signal remains present in the acoustic signal 182 received by the one or more processors. The absence of a heart signal could indicate that another physiological state should be determined (or that the user is not present on the cushioning layer 40), such as death of the user as described in relation to FIG. 26 below.

Changes in heart rate are possible during irregularity in breathing. For example, heart rate could increase to compensate for lowered oxygen levels in the blood.

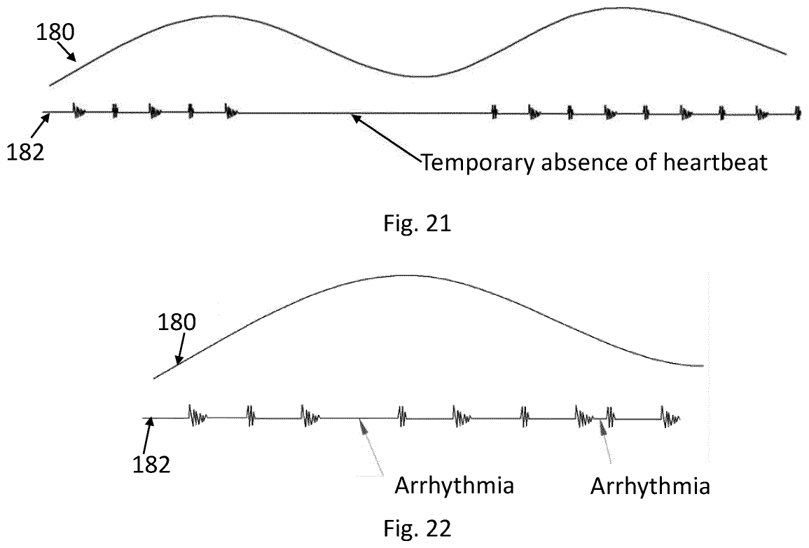

FIG. 21 shows a graphical representation of pressure 180 and acoustic signals 182 showing an interruption in heartbeat of the user. An interruption in heartbeat includes stoppages or shallowness in heartbeat. An interruption in heartbeat is determined in response to a detection of a temporary absence of the heart signal of the user detected in the acoustic signal 182 for a predetermined duration.

An interruption in heartbeat of the user is only determined by the one or more processors if a respiration signal remains present in the acoustic signal 182 received by the one or more processors. The absence of a respiration signal could indicate that another physiological state should be determined (or that the user is not present on the cushioning layer 40).

FIG. 22 shows a graphical representation of pressure 180 and acoustic signals 182 showing an irregularity in heartbeat of the user. An irregularity in heartbeat includes changes in frequency of heartbeat. An irregularity in heartbeat (e.g. heart arrhythmia) is determined in response to a detection of an irregular change in the heart rate of the user. An irregular change in the heart rate may be the heart rate having a greater than threshold change or variation in the heart rate. For instance, heart rate variability may be determined and an irregular change in heart rate may be detected in response to determining that the heart rate variability exceeds a threshold heart rate variability.

For example, an irregular change in heart rate of the user can be an increase heart rate of the user above the threshold heart rate variability, which is a sign of atrial fibrillation potentially causing thrombosis to the user.

During deep sleep state of the user, the variability of heart rate should normally stay within approximately 5%, as well as the separation of S₁ and S₂ heart signals. Variabilities in heart rate detected in the acoustic signal 182 can be analysed to detect possible heart arrhythmia. Some variability is associated with the person breathing in or out, which can be taken into account using the pressure signal 180.

An irregularity in heartbeat from arrhythmia can also be determined in response to a detection of additional heartbeats between the separation of S₁ and S₂ heart signals. An irregularity in heartbeat from arrhythmia can also be determined in response to a detection of irregular change in a heartbeat of the user, despite the user having a regular and healthy heart rate. For example, an irregular change in heartbeat of the user can be abnormal fluctuations in amplitude of the heartbeat, which is a sign of sinus bradycardia.

An irregularity in heartbeat of the user is only determined by the one or more processors if a respiration signal remains present in the pressure signal 180 received by the one or more processors. The absence of a respiration signal could indicate that another physiological state should be determined (or that the user is not present on the cushioning layer 40).

In addition or alternatively, a machine learning model can be applied to the acoustic signal or the cardiac signal to identify irregular heartbeats (e.g. through classification). This can be trained on labelled training data showing irregular and regular cardiac signals.

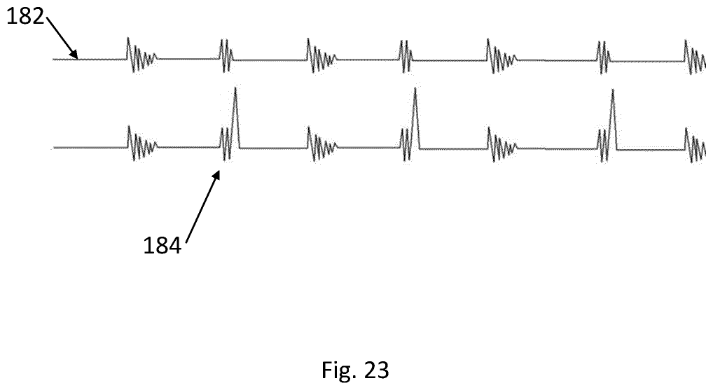

FIG. 23 shows a graphical representation of abnormal sounds synchronous with heartbeat 184 of the user.

It is possible to detect abnormal sounds that are synchronous with heartbeat 184. These sounds may originate from the heart itself, and can be an indication of abnormal heart condition. Abnormal sounds may also originate from blood flowing in a wrong direction from blockage of blood vessels or physical defects in the heart itself. Some of the heart sounds are audible depending on whether the person breathes in or out, which can be determined from the pressure signal 180.

Abnormal sounds synchronous with heartbeat 184 by the user may be determined if an abnormal sound is sensed by the microphone and repeats at the same, or similar, frequency as (synchronous with) the user's heart rate and has the same or similar position within the cardiac cycle. Having said this, the abnormal sound synchronous with heartbeat 184 need not occur in every cardiac cycle. Instead, it can be sufficient to detect a repeated abnormal sound within a set number or proportion of cardiac cycles. An abnormal sounds synchronous with heartbeat 184 does not have to have the exact same frequency as the user's heart rate, there can be some variability.

Abnormal sounds synchronous with heartbeat 184 can be constant or transient. Transient abnormal sounds synchronous with heartbeat 184 can be detected by comparison to a long term average heart sound pattern. Constant abnormal sounds synchronous with heartbeat 184 can be detected by comparing against a database of heart sounds. For example, users' heart signals are stored and classified, for example by machine learning methods, and constant abnormal sounds synchronous with heartbeat 184 can be detected by comparison against the database.

The extraction of the heart signal allows the determination of the phase, or phase range(s), in which the abnormal sound occurs within a cardiac cycle of the user. Accordingly, an abnormal sound may be determined to be synchronous with heartbeat in response to determining one or more repetitions across heartbeats that occur within the same cardiac phase (or cardiac phase range) within their respective heartbeats.

Examples of potential abnormal sound synchronous with heartbeat 184 is heart murmurs, galloping, clicks and rubs; although other abnormal sounds may exist related to other cardiac irregularities.

FIG. 24 shows a graphical representation of pressure 180 and acoustic signals 182 showing abnormal sounds synchronous with breathing 186 of the user. Abnormal sounds synchronous with breathing 186 by the user may be determined if an abnormal sound is sensed by the microphone and repeats at the same frequency as (synchronous with) breathing. Having said this, the abnormal sound synchronous with breathing 186 need not occur in every respiratory cycle. Instead, it can be sufficient to detect a repeated abnormal sound within a set number or proportion of respiratory cycles.

The co-use of a pressure signal 180 and an acoustic signal 182 enables determination of the phase, or phase range(s), in which the abnormal sound occurs within a respiratory cycle of the user. Accordingly, an abnormal sound may be determined to be synchronous with breathing in response to determining one or more repetitions across respiratory cycles that occur within the same respiratory phase (or respiratory phase range) within their respective respiratory cycles.

Abnormal sounds synchronous with breathing include, but are not limited to, snoring 188, wheezing, crackling, stridor, rhonchi and pleural rubs. These abnormal sounds are an indication of various disease conditions and abnormal physiological states of the user, examples include obstruction of airways, asthma, pneumonia, bronchitis, inflammation, air or fluid in or around the lungs, chronic obstructive pulmonary disease, bronchiectasis and particularly any other obstructive pulmonary diseases that causes abnormal sounds in the lungs.

FIG. 25 shows another graphical representation of pressure 180 and acoustic signals 182 showing abnormal sounds synchronous with breathing of the user, wherein the abnormal sounds are snoring 188.

Snoring 188 and other abnormal sounds synchronous with breathing 186 differ from each other depending on various properties of the acoustic signal 182 at which the abnormal sounds synchronous with breathing 186 is exhibited. This can be dependent on, amongst other things, on the phase (or phase range) within the respiratory cycle of the respiration signal at which the abnormal sounds synchronous with breathing 186 is detected, amplitude and duration of the abnormal sounds synchronous with breathing 186 and spectral composition of the abnormal sounds synchronous with breathing 186. Snoring is primarily associated with being audible during the breathing in phase range of the respiratory cycle. Snoring has an amplitude that is usually greater and duration that is usually longer than a typical breathing out sound during sleep of the user. Snoring may have a particular spectral composition associated with it.

Having said this, abnormal sounds synchronous with breathing 186 will inevitably vary between each user and therefore abnormal sounds synchronous with breathing 186 may be compared against a database including acoustic parameters of abnormal sounds synchronous with breathing 186, such as the ones listed above (phase, amplitude, duration and spectral composition). As with the detection of other abnormal sounds discussed herein, the detection of abnormal sounds synchronous with breathing can be achieved through the use of one or more machine learning models.

FIG. 26 shows a graphical representation of a pressure 180 and acoustic signals 182 showing death of the user. The co-use of the pressure signal 180 and the acoustic signal 182 can determine death of the user on the cushioning layer 40. Commonly, if a person dies, the heartbeat will stop, which is indicated by the disappearance of the heart signal in the acoustic signal 182. Meanwhile, the respiration signal in the pressure signal 180 will persist for an extended time after absence of the heart signal. Accordingly, death is determined in response to a detection of an absence of the heart signal whilst the respiration signal remains present for a predefined time interval. Consequently, after the determination of death of the user, an alert or alarm may be issued to a third party, for example a caregiver.

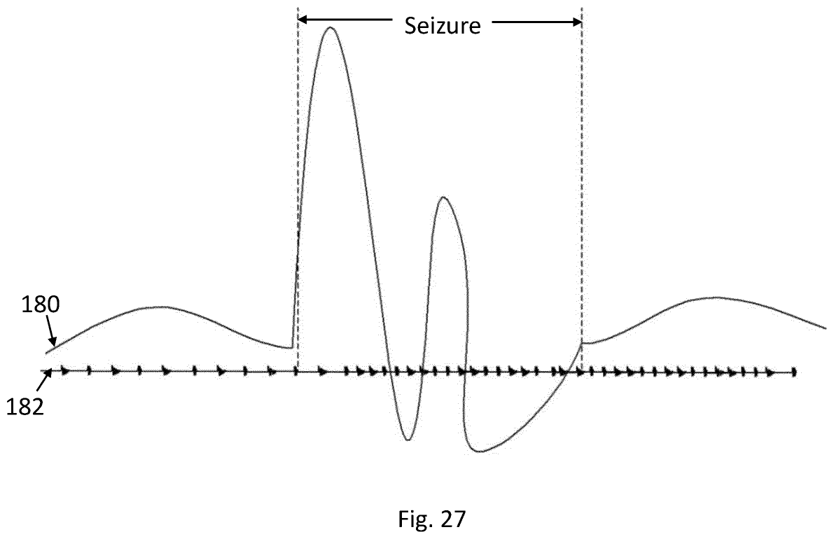

FIG. 27 shows a graphical representation of pressure 180 and acoustic signals 182 showing a seizure of the user. For example, during an epileptic seizure, muscle contractions and shaking occur commonly. The shaking movements can involve part or whole of the body and can last from several seconds to several minutes. These abrupt movements and shaking can be detected from the pressure signal 180. The heart rate can increase during the seizure, detectable from the microphone signal. Accordingly, embodiments detect a seizure in response to the amplitude of the pressure signal 180 exceeding a seizure pressure threshold and the heart rate determined from the audio signal 182 exceeding a seizure heart rate threshold.

In one embodiment, a seizure is only detected if the seizure pressure threshold and the seizure heart rate threshold are exceeded for a predetermined period of time. An alert or alarm may be issued to a third party, such as a caregiver, in response to the detection of a seizure to alert the caregiver to the potential need for treatment.

In one embodiment, in response to a seizure being detected and to remove noise due to movement of the user, the acoustic signal may be amplified and/or filtered further to provide continued detection of the user's cardiac signal. For example, the frequency capture range of the acoustic signal may be narrowed to a range closer to the frequency response of the heartbeat sound. The acoustic signal may undergo upward and/or downward compression depending on the nature of the filtered frequency response of the acoustic signal.

Sounds originating from the heart are generally within the 4-70 Hz range. All heart related acoustic vibrations, including abnormal sounds, are within 0.1-200 Hz. However, in some instances a narrower frequency range may be implemented. For example, if only the user's heart rate is to be determined, the frequency range can be as narrow as 5-12 Hz. This can be implemented in the example as described above in relation to the user having a seizure. A narrower and more specific frequency range removes noise associated with signals outside the narrower frequency range. Accordingly, the frequency range for detecting the cardiac signal can be selected from 0-15 Hz, 5-15 Hz, 0-70 Hz, 5-70 Hz, 0-150 Hz and 0-200 Hz.

Bruxism during sleep can be detected by the physiological monitoring system. Its cause is often associated with stress, anxiety and sleeping disorders, such as sleep apnea. Symptoms of bruxism vary from mild pain in the facial area to severe wearing of teeth.

Teeth grinding noise can be detected from the acoustic signal 182. Movement of the jaw can be detected in the pressure signal 180. Relationship between bruxism and other physiological conditions and disorders can be made in parallel with the detection of sleep states, vital signs and sleep disorders, as bruxism is frequently a result of stress, anxiety, snoring, sleep apnea, sleep talking, sleep time restless movement, alcohol and drug use, and excessive drinking of caffeinated drinks—all of which are accompanied by physiological parameters detectable with the sensor system (e.g. elevated heart rate and breathing rate, movements and noises).

Teeth grinding noises and jaw movement noises can be detected from their characteristic spectral compositions. A machine learning classifier could be trained to detect the presence of both teeth grinding noises in the acoustic signal 182 and jaw movement noises in the pressure signal 180.

Teeth grinding noises and jaw movement noises will also vary between each user and therefore may be classified based on a database including acoustic parameters indicative of teeth grinding noises and jaw movement noises to detect the presence of bruxism in the user.

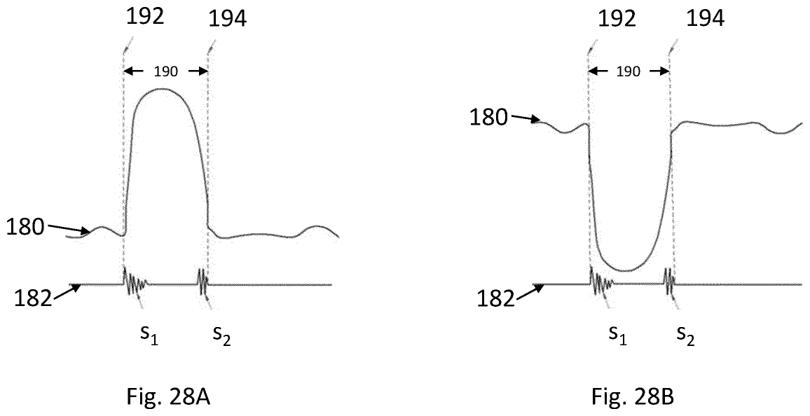

FIG. 28A and FIG. 28B show graphical representations pressure 180 and acoustic signals 182 indicative body positions of the user. The graphical representation shows the pressure signal 180 and the acoustic signal 182, wherein the pressure signal 180 shows a change in amplitude with respect to time between two points represented along the x axis of the acoustic signal 182.

The one or more processors are further configured to detect a cardiac pressure signal of the user in the pressure signal 180. The cardiac pressure signal, detected from the pressure signal 180, is in addition to the cardiac signal from the acoustic signal 182. The cardiac pressure signal can be extracted from the overall pressure signal 180, or from the respiration signal, for instance through the use of a high pass filter to filter out the respiratory changes in pressure.

In determining a body position of the user, the signal from the acoustic sensor is used as a trigger to monitor the pressure signal 180. The movement of the heart is detected in the acoustic signal picked up by the microphone, which is also accompanied by a corresponding small change in the pressure signal. However, the pressure signal change originating from the heart movement is much smaller than that caused by breathing. Depending on the body position of the person resting on the cushioning layer 40, the shape of the pressure signal 180 from heart movement varies. From the analysis of the shape of the pressure signal 180 during a measurement window 190, the body position of the user lying on the cushioning layer 40 can be determined.

Specifically, there are several distinctive heart sounds represented in the acoustic signal 182. Most prominent are the first heart sound (S₁) and second heart sound (S₂). The S₁ heart sound is a sound indicating the beginning of the systole phase of the cardiac cycle. The S₁ sound is caused by turbulence created within the heart when the mitral and tricuspid valves close. The S₂ sound is a sound indicating the end of the systole phase and the beginning of the diastole phase. The S₂ sound is caused by the closure of the aortic and pulmonic valves. Between the occurrence of S₁ and S₂, the right ventricle movement causes changes in the pressure signal 180. By making use of the S₁ and S₂ sounds to initiate the acquisition of the cardiac pressure signal, the cardiac pressure signal can be better extracted from noise within the overall pressure signal 180.

The phase/direction of the change in pressure indicates the body position of the user. For example, when the user is lying on one side, the movement of right ventricle causes a rising pressure signal 180; whereas when the person is lying on another side, the movement of right ventricle causes a falling pressure signal 180. The rising pressure signal 180 is at a local maximum during the systolic phase of the cardiac cycle and the falling pressure signal 180 is at a local minimum during the systolic phase of the cardiac cycle.

The S₁ sound can be used as a trigger to initiate the measurement window 190 at a start measurement time 192 and the S₂ sound can be used as a trigger to end the measurement window 190 at an end measurement time 194. Analysis of the pressure signal 180 between the two start 192 and end measurement times 194 provide information about the position of the heart compared to the surface of the cushioning layer 40, thus the body position of the user may be determined.

In an example, particularly amongst healthy individuals with normal anatomy, the user lying on their left side of the body corresponds to a rising pressure signal after the S₁ sound and the user lying on their right side of the body corresponds to a falling pressure signal after the S₁ sound.

Actuation of the heart valve operation and their movement direction can be identified through machine learning algorithms to improve the determination of a body position of the user.

In some embodiments, due to the variability in heart position and heart movements between users, it is important to calibrate the system for monitoring one or more physiological states for each user. For example, a user with an abnormal physiological condition such as dextrocardia may have a different pressure signal response corresponding to the movement of the user's heart. Accordingly, the system may be calibrated to consider this difference.

For example, in the case of the user having dextrocardia, or other conditions that may affect the user's heart position and/or heart movement, it may be advantageous to calibrate the user lying on their left side of the body to correspond to a falling pressure signal after the S₁ sound and the user lying on their right side of the body to correspond to a rising pressure signal after the S₁ sound. This can be determined by instructing the user to lie on their left side and taking measurements, and then instructing the user to lie on their right side and taking measurements. The system can then determine which signal profile (e.g. rising or falling pressure) is correlated with which body position.

Furthermore, the body position of the user can be logged and, depending on the history of the user's body positions, the user, or a caregiver, can be alerted that the body position of the user has remained unchanged for a predetermined duration of time. This feature, for example, could be implemented to prevent the formation of pressure ulcers amongst patients.

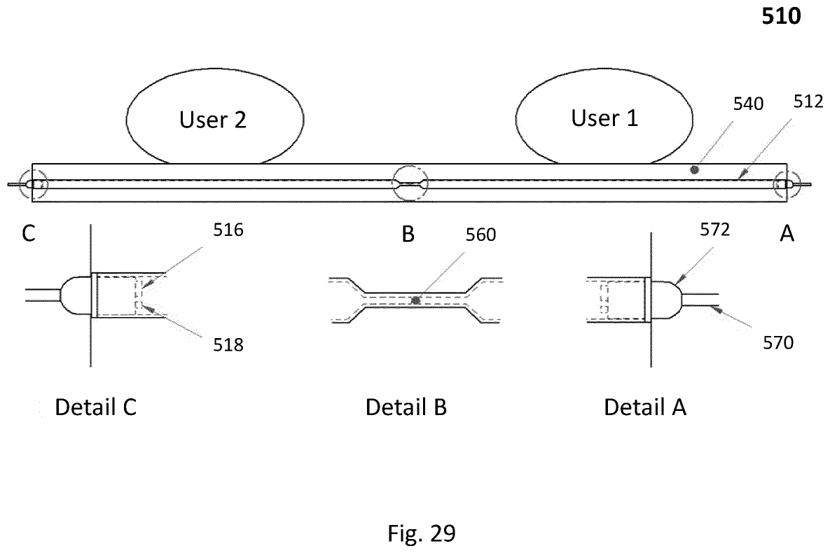

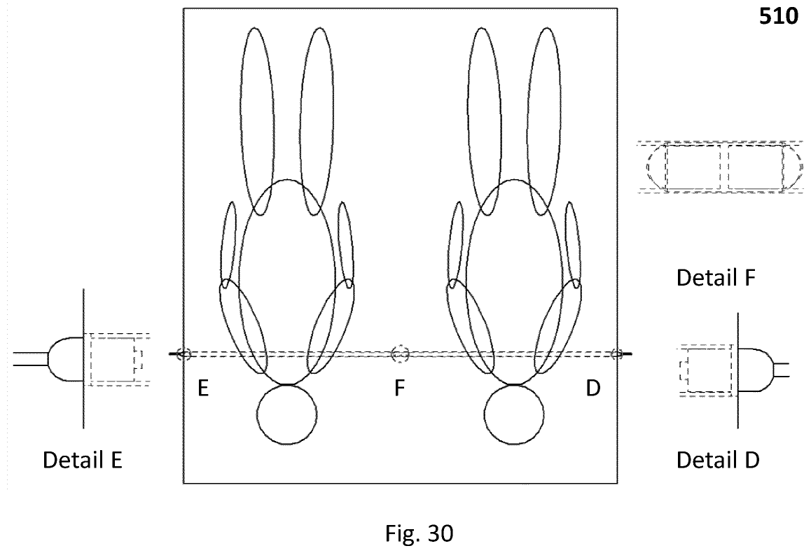

FIG. 29 shows the sleep monitoring apparatus being used in a multiple user operation mode 510. FIG. 30 shows a plan view of the sleep monitoring apparatus being used in a multiple user operation mode.

The sleep monitoring apparatus 10 of the system for monitoring one or more physiological states can be configured for multiple user operation 510. Two users on a single measurement surface (e.g. in a two-person bed) can be monitored simultaneously using two different configurations. Specifically, one configuration involves installing two separate sensor systems in the cushion layer 540 and the other configuration involves installing the sensor system with one signal collecting tube 512 extending across the whole width of the cushioning layer 540, as shown in FIG. 29 and FIG. 30.

In case of the separate sensor systems, the sleep monitoring apparatus 10 is installed on each user's respective sides (two in this instance), with each signal collecting tube 12 extending to about half of the width of the cushioning layer.

For the single tube 512 solution, the arrangement comprises of one long signal collecting tube 512 extending across the whole width of the cushioning layer 540. The sleep monitoring apparatus 510 in this configuration comprises two individual controllers with an acoustic sensor 516 and a pressure sensor 518 (detail A and C in FIG. 29, and detail D and E in FIG. 30) and a separator plug 560 to divide the tube 512 into two independent compartments (detail B in FIG. 29 and detail F in FIG. 30).

The data collected by the two independent controllers can be shared with one another or with a shared computing system for analysis. The separation of the sensor systems or the use of a separator plug 560 eliminates signal artefacts originating from the other user and allows both user information to be taken into account in any further analysis. Utilizing a single tube 512 that is sealed at the center provides a more cost effective and easy to manufacture solution relative to the provision of two separate systems.

The systems described herein can monitor sleep state (level of consciousness) and issue instructions to external devices to control the devices based on the sleep state. For instance, potentially disruptive devices, such as those that emit sound or light when active, can be turned off during the times at which the user is in a particular sleep state (e.g. REM sleep) and turned on when the user's level of consciousness is at a different level/sleep state (e.g. awake, deep sleep, etc.). The sleep state/level of consciousness can be determined based on the heart rate and respiratory rate, with higher rates indicating higher levels of consciousness and lower rates indicating lower levels of consciousness.

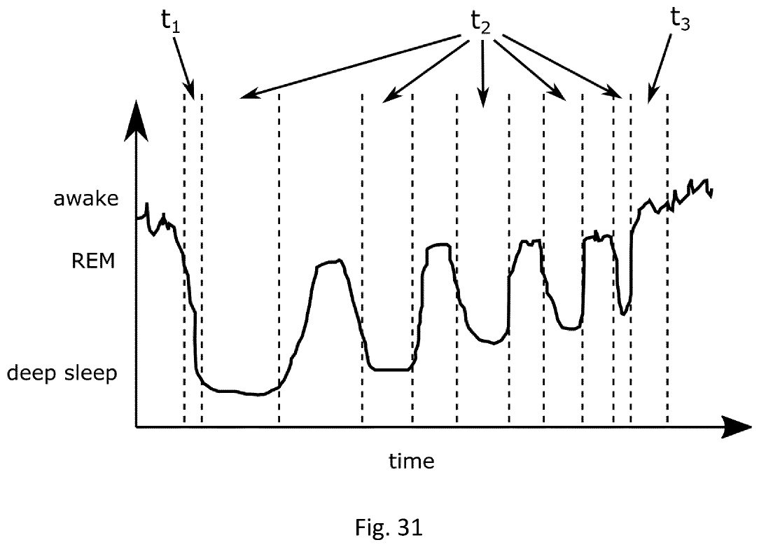

FIG. 31 shows a plot of level of consciousness over time, divided into various sleep states. Various sleep states are indicated via shaded regions.

Data on the user's heartbeat and/or breathing can be used to construct a sleep curve describing changes in the sleep state of the user against time. This real-time knowledge of the changes in the sleep state of the user with respect to time can be used by the physiological state monitoring system to send control signals to external devices, in particular smart devices at home.

For example, external devices include, but are not limited to, household devices, such as: fans, thermostats (of heating/cooling system), audio amplifiers, kettles, air filters, air conditioners and fridges. Control signals may be used to control an operating parameter of one or more of the external devices. For example, the physiological state monitoring system may send a control signal to turn on or off one or more of the external devices, or adjust one or more settings of the one or more external devices, depending on the past, present and/or future sleep state(s) of the user.

Some household devices would be useful or not disruptive for a user that is in deep sleep, but disruptive for the user during light and REM sleep due to noise or light emitted, for example an air purifier or air conditioner. To minimize the chances of waking up the user or otherwise having the quality of sleep degraded, this could be controlled by the control signals.

A sleeping user is least alert during deep sleep state, during which time the disruptive effects of noises are least pronounced. The sensor system can determine the sleep state of the user and send a control signal to the external device to indicate that it is acceptable to turn on.

Some devices could be switched on when the person wakes up. Some appliances, for example an audio amplifier, would be desirable to be switched off when the user has fallen asleep.

Furthermore, a smart/soft wake up system could be implemented depending on the sleep curve of the user. The user may forget to switch off the lights or music when before falling asleep. This can also be done automatically by the control signal from the sensor system. Additionally, certain external devices can be turned on based on the sleep state of the user that is determined. For example, a kettle or a pot of water in the kitchen can be boiled when the person wakes up in the morning. Certain external devices can be controlled continuously, for example a thermostat of a heating/cooling system to provide optimal sleep quality, as determined by the sleep monitoring system. In this case, each sleep state may be associated with a given setting, for instance, a given temperature of the thermostat.

For example, in FIG. 31, schedule for control signals to external devices are split up into three distinct sleep state and/or transition categories: t₁, t₂ and t₃. During t₁, all external devices that may disrupt the user's sleep are turned off whilst the user is transitioning from an awakened state to REM sleep state and finally to a deep sleep state. This transition can be detected based on the starting level of consciousness (awake—level of consciousness above a corresponding threshold) and the gradient of the sleep curve. At t₂, external devices that are useful during, but external devices that could potentially be disruptive whilst the user is awake or in REM sleep are switched on. This is because whilst the user is in the deep sleep state, the user will be unresponsive to disruptive noises from external devices and/or the environment. The user can be determined to be in deep sleep when their level of consciousness is below a certain threshold. During t₃, the user transitions from the deep sleep state to REM sleep state and to an awakened state, or alternatively, directly from a REM sleep state to the awakened state. During t₃, the above mentioned smart/soft wake up system is implemented, wherein, for example, devices that are desirable to be used when awake (or that are disruptive, and therefore must be kept off during sleep) are switched on such as a kettle being sent a control signal to boil water when the user is awake in the morning.

In each case, the sleep monitoring system is programmed with rules regarding one or more instructions to be issued for particular sleep states. Each rule specifies as last on instruction to a certain external device or devices. Each rule may be transmitted according to a network or other connection to the external device, such as wireless network (e.g. Wi-Fi network or Bluetooth connection).

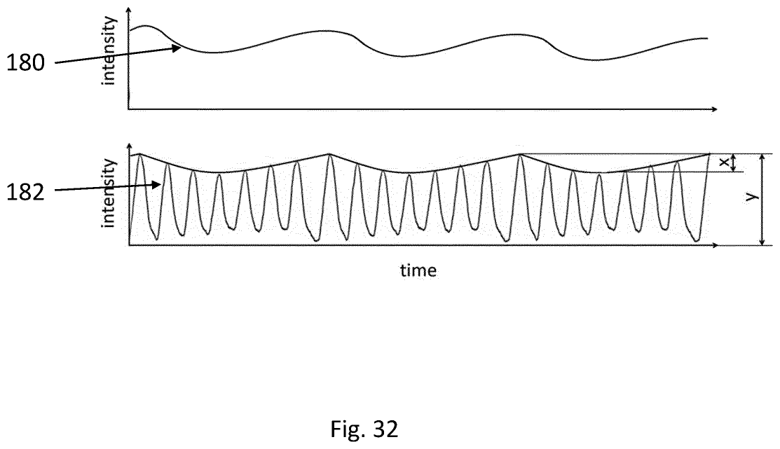

FIG. 32 shows a graphical representation of pressure and acoustic signals indicative of the user's level of hydration.

The level of hydration of the user can be estimated and monitored using information about breathing rate and heart rate signals detected in the pressure signal 180 and acoustic signal 182 respectively.

Significant fluctuations in heart signal amplitude across a respiratory cycle indicates a lower fluid level within the user's cardiovascular system.

For users with heart conditions, it is essential that fluid levels are kept within an acceptable range. Accordingly, the system can monitor the changes in blood pressure across a respiratory cycle to ensure that it is in an acceptable range. This monitoring can be optimized by keeping the person in constant state of slight thirst (helpful for users with heart conditions). In order to assess whether fluid should be administered (e.g. through the user drinking or through an intravenous drip), the fluid level in the body has to be monitored.

The top half of FIG. 32 represents the user's heart signal and the bottom half represents the user's breathing signal. Generally, for healthy individuals, the heartbeat signal amplitude change (x) over the respiratory cycle should to be within about 10% of the total heart signal amplitude (y). For people with heart failure, sometimes referred to as congestive heart failure, it is particularly important that their fluid levels are monitored and maintained within this range.

As shown in FIG. 32, the heart signal varies not only across each heartbeat, but also across each respiratory cycle. The change in amplitude over a respiratory cycle (x) should be within a certain threshold (in this case, 10%) of the total amplitude of the signal across a full respiratory cycle.

Each cardiac cycle includes a maximum peak across the cardiac cycle (relating to systolic contraction). The height of this peak will depend on the relative phase of the respiratory cycle. In order to determine the change in amplitude, the lowest peak across a respiratory cycle is subtracted from the highest peak across that respiratory cycle. In order to determine the total heart signal amplitude, the minimum signal across the respiratory cycle is subtracted from the highest peak for that respiratory cycle. The ratio of the change (x) to the total amplitude (y) is then determined.

One or more thresholds for the ratio can be set and, when these thresholds are exceeded, then an alert may be issued. For instance, values of x above 10% of y indicate pathology. On the other hand, sometimes water in the body starts to accumulate in the lungs or other organs, in which case it is desirable to keep the body in larger water deficit, in this case x has to be within about 20% of y.

The analysis of the two signals (heartbeat and breathing) enables monitoring of the water amount in the body and, based on the need, issue an alert in instruction to adjust the user's fluid level.

Whilst the embodiments described herein are not limited to signal processing with reference to implementation of acoustic 182 and pressure signals 180 from the sleep monitoring apparatus 10, it is possible to be implemented in other physiological monitoring apparatuses.

Information on the determination of the user's physiological parameters and states can be used to significantly improve the accuracy of determining future or concurrent physiological states of the user. It is also noted that the physiological monitoring system could be part of a network of physiological monitoring systems to help improve the determination of physiological states of the user using data analytic methods.

Advantageously, the present invention allows data to be acquired and analysed from a calm, uninterrupted state in the user's natural environment. The physiological information does not have to be collected in a controlled environment, at a specified period (for example, a doctor's appointment). For example, abnormal heart sounds can be more accurately detected if the measurements are done during when the user is calmly lying on the bed, for example during deep sleep time. This enables higher quality and noise free data to be selected for further analysis. Additionally, unnatural environments can induce additional stress or anxiety, which is detrimental for some measurements, such as sleep disorder evaluation.

While certain arrangements have been described, the arrangements have been presented by way of example only, and are not intended to limit the scope of protection. The inventive concepts described herein may be implemented in a variety of other forms. In addition, various omissions, substitutions and changes to the specific implementations described herein may be made without departing from the scope of protection defined in the following claims.

Embodiments can be provided in accordance with the following clauses.

First Clauses:

1. A sleep monitoring system for monitoring and measuring sleeping activity comprising:

a mattress or a seat (1) into which are arranged a fluidic (gases based), non-electrical elastic material made signal collectors (2) for mechanical vibrations under the sleeping surface (3) in order to collect and bring mechanical vibration signals out from the mattress for the purpose of detection by sensors (4), which are connected to the part of signal collector that remain outside of the mattress or which are put into place where they remain outside the direct influence of sleeping persons or at least 5 cm away from the sleeping persons.

2. The sleep monitoring system according to clause 1 in any kind of geometrical shape, characterized by that the signal collector is assembled from at least one elastic material made, gas filled vessel (5).

3. The sleep monitoring system according to clause 2 characterized by that the gas filled elastic vessel is a pipe made of elastic polymeric material such as rubber or PDMS.

4. The sleep monitoring system according to clauses 2 or 3 characterized by that the signal collector is a pipe filled with gas selected from air, N2, Ar etc.

5. The sleep monitoring system according to clauses 3 or 4 characterized by that different geometrical shape cross-section pipes are used as signal collectors, including circular or flat ones.

6. The sleep monitoring system according to clause 4 characterized by that the pipe as signal collector is used in the manner where one end of the pipe is closed and air pressure sensor for signal detection is connected with other end of the pipe.

7. The sleep monitoring system according to clause 1 characterized by that a small holes or air channels are made to the signal collector or porous wall materials are used for its controlled exchange of atmospheres outside and inside of the elastic vessel of collector.

8. The sleep monitoring system according to any of the preceding clauses characterized by that sensors are firmly connected to signal collector that remain outside the mattress.

9. The sleep monitoring system according to any of the preceding clauses characterized by that signal collector system is integrated to the seat of a chair.

Further embodiments can be provided in accordance with the following additional clauses.

Second Clauses:

1. A system for use in monitoring one or more physiological parameters of a user, the system comprising:
at least one sensing unit comprising at least one vibration sensor; and
at least one elastic tube configured to be embedded within a cushioning layer for supporting at least a portion of the user, wherein one end of the at least one tube is sealed and the other end of the at least one tube is closed by the at least one sensing unit so as to form a volume that is filled with fluid and that is defined by one or more inner walls of the tube and a surface of the at least one sensing unit,
wherein the at least one elastic tube is configured to transmit vibrations from one or more sections of the at least one tube to the at least one sensing unit for detection by the at least one vibration sensor.

2. The system of clause 1, wherein the at least one vibration sensor comprises one or more of a microphone and a pressure sensor.

3. The system of clause 1 or clause 2, wherein the at least one vibration sensor operates at a frequency capture range from 0-200 Hz.

4. The system of any preceding clause wherein the at least one elastic tube has a uniform cross-section along its length.

5. The system of any preceding clause wherein the at least one elastic tube comprises one or more indents along the one or more inner walls of the tube to help prevent the tube sticking shut when crushed.

6. The system of clause 5 wherein the one or more indents are in the form of one or more ridges running along the length of the tube.

7. The system of any of clauses 5 or 6 wherein the one or more indents comprise a plurality of evenly spaced indents around the circumference of the tube.

8. The system of any preceding clause, wherein the at least one elastic tube comprises a plurality of elastic tubes that are connected to a common node to form a single volume for transmitting vibrations back to the sensing unit.

9. The system of any preceding clause, wherein the system further comprises a controller configured to determine one or more of:
one or more physiological parameters of the user from detected vibrations; and
one or more physiological states of the user from detected vibrations.

10. The system of clause 9, wherein the controller forms part of an external device and wherein the at least one sensing unit is configured to send data relating to the detected vibrations to the external device for processing by the controller.

11. The system of clause 9, wherein the controller forms part of the sensing unit.

12. The system of any of clauses 9 to 11, wherein the controller is configured to determine, based on the detected vibrations, one or more of a heart rate of the user, a respiration rate of the user, a body position of the user and movement of the user.

13. The system of any of clause 12 wherein the controller is configured to determine the heart rate of the user, wherein determining the heart rate comprises:
filtering the detected vibrations to form filtered vibration data over a predefined frequency range;
detecting local maxima in the filtered vibration data; and
determining the heart rate based on the detected local maxima.

14. The system of clause 12 or clause 13 wherein the controller is configured to determine the respiration rate of the user, wherein determining the respiration rate comprises:
detecting local maxima in the detected vibrations; and
determining the respiratory rate based on the detected local maxima.

15. The system of any of clauses 12-14 wherein the controller is configured to determine movement of the user based on the detected vibrations, wherein determining movement of the user comprises determining whether an amplitude of the detected vibrations exceeds a predefined amplitude threshold.

16. The system of any of clauses 12-15, wherein:

the at least one vibrations sensor comprises a microphone configured to generate a microphone signal based on the detected vibrations and a pressure sensor configured to generate a pressure sensor signal based on the detected vibrations; and the controller is configured to determine the body position of the user, wherein the body position of the user is determined based on phase differences in the microphone and pressure sensor signals.

17. The system of any of clauses 12-16, wherein the controller is configured to determine a sleep state of the user based on one or more of the heart rate of the user, the respiration rate of the user and movement of the user.

18. The system of clause 17 wherein the controller is configured to perform one or more of the following:
determine that the user is awake in response to determining that one or both of the heart rate and respiration rate are above a corresponding first threshold and variation in one or both of the heart rate and respiration rate is below a corresponding variation threshold;
determine that the user is in rapid eye movement, hereinafter referred to as REM, sleep in response to determining that one or both of the heart rate and respiration rate are above the corresponding first threshold and the variation in one or both of the heart rate and respiration rate is above the corresponding variation threshold;
determine that the user is in light sleep in response to determining that one or both of the heart rate and respiration rate are below the corresponding first threshold and above a corresponding second threshold that is less than the corresponding first threshold; and
determine that the user is in deep sleep in response to determining that one or both of the heart rate and respiration rate are below the corresponding second threshold.

19. The system of clause 17 or clause 18, further comprising an alarm system that is configured to issue an alarm in response to a determination that a predefined sleep state or predefined change in sleep state has been reached during an alarm period between a predefined start alarm time and a predefined end alarm time.

20. The system of clause 19 wherein the alarm system is configured to perform one or more of the following:
issue the alarm in response to a determination that the user has transitioned from REM sleep to light sleep during the alarm period;
issue the alarm in response to a determination that a current time is within a predefined period from an end alarm time and the user is in light sleep during the alarm period, the predefined period being shorter than the alarm period; and
issue an alarm in response to the end alarm time being reached and the predefined sleep state or predefined change in sleep state has not been detected during the alarm period.

21. A computer system comprising one or more processors configured to:
receive one or more vibration signals indicative of vibrations detected within a cushioning layer for supporting at least a portion of the user; and
determine, based on the one or more vibration signals, one or more of a heart rate of the user, a respiration rate of the user, a body position of the user and movement of the user.

22. The computer system of clause 21 wherein the one or more processors are further configured to determine a sleep state of the user based on one or more of the heart rate of the user, the respiration rate of the user and movement of the user.

23. The computer system of clause 22 wherein the one or more processors are further configured to issue an alarm in response to a determination that a predefined sleep state or predefined change in sleep state has been reached during an alarm period between a predefined start alarm time and a predefined end alarm time.

24. A kit of parts for use in monitoring one or more physiological parameters of a user, the kit of parts comprising:
at least one sensing unit comprising at least one vibration sensor; and
at least one elastic tube configured to be embedded within a cushioning layer for supporting at least a portion of the user, wherein one end of the at least one tube is sealed and the other end of the at least one elastic tube open for receiving the at least one sensing unit,
wherein the kit of parts is configured such that when the at least one sensing unit is received within the other end of the at least one elastic tube, a volume is defined by one or more inner walls of the tube and a surface of the at least one sensing unit for containing fluid such that the at least one elastic tube is configured to transmit vibrations from one or more sections of the at least one tube to the at least one sensing unit for detection by the at least one vibration sensor.

1. A system for use in monitoring one or more physiological states of a user, the system comprising one or more processors configured to:
receive a pressure signal representing pressure within a cushioning layer supporting at least a portion of a user and an acoustic signal representing acoustic vibrations within the cushioning layer; and
determine, based on the pressure signal and acoustic signal, the one or more physiological states of the user.

2. The system of claim 1, wherein the pressure signal represents pressure oscillations at a lower frequency than the acoustic vibrations.

3. The system of claim 1, wherein the one or more physiological states of the user comprise at least one of:
a body position of the user;
a level of hydration of the user; or
one or more abnormal physiological states of the user.

4. The system of claim 1, wherein one or both of:
the pressure signal comprises a respiration signal indicative of respiration of the user and the determination of the one or more physiological states of the user is based on the respiration signal; and
the acoustic signal comprises a cardiac signal indicative of one or more cardiac cycles of the user and the determination of the one or more physiological states of the user is based on the cardiac signal.

5. The system of claim 1, wherein the pressure signal comprises a respiration signal indicative of respiration of the user, the determination of the one or more physiological states of the user is based on the respiration signal, the one or more physiological states of the user comprise one or more abnormal physiological states of the user determined based on changes in the respiration signal, and the one or more abnormal physiological states of the user include at least one of:
an interruption in breathing determined in response to a detection of an absence of change in the respiration signal for a predetermined duration; or
an irregularity in breathing pattern determined in response to a detection of irregular changes in respiration rate of the respiration signal.

6. (canceled)

7. (canceled)

8. (canceled)

9. The system of claim 1, wherein the acoustic signal comprises a cardiac signal indicative of one or more cardiac cycles of the user, the determination of the one or more physiological states of the user is based on the cardiac signal, the one or more physiological states of the user comprise one or more abnormal physiological states of the user determined based on changes in the cardiac signal, and the one or more abnormal physiological states include at least one of:
an interruption in heartbeat determined in response to a detection of an absence of change in the cardiac signal for a predetermined duration;
an irregularity in heartbeat determined in response to a detection of irregular changes in heart rate of the cardiac signal; or
presence of one or more abnormal sounds synchronous with heartbeat of the user.

10. (canceled)

11. (canceled)

12. (canceled)

13. The system of claim 1, wherein the pressure signal comprises a respiration signal indicative of respiration of the user, the acoustic signal comprises a cardiac signal indicative of one or more cardiac cycles of the user, the determination of the one or more physiological states of the user is based on the respiration signal and the cardiac signal, the one or more physiological states of the user comprise one or more abnormal physiological states of the user determined based on changes in the pressure signal and acoustic signal, and the one or more abnormal physiological states include at least one of:
presence of one or more abnormal sounds synchronous with breathing;
death;
seizure; or
bruxism.

14. The system of claim 13, wherein the determination of the one or more physiological states of the user based on the respiration signal and the cardiac signal comprises the determination of at least one of:
death in response to a detection of an absence of the cardiac signal or an absence of changes in the cardiac signal whilst the respiration signal continues to be detected or changes in the respiration signal continue to be detected for a predefined time interval; or
seizure in response to a detection that the pressure signal or respiration signal exceeds an amplitude threshold and a frequency of the acoustic signal or cardiac signal exceeds a frequency threshold.

15. (canceled)

16. The system of claim 1, wherein determining the one or more physiological states of the user comprises:
obtaining an input derived from one or both of the pressure signal and acoustic signal;
inputting the input into a machine learning model configured to determine, for each of a set of potential physiological states, a probability that the user has the corresponding physiological state; and
determining the one or more physiological states based on each probability output from the machine learning model.

17. The system of claim 12, wherein the pressure signal comprises a respiration signal indicative of respiration of the user, the acoustic signal comprises a cardiac signal indicative of one or more cardiac cycles of the user, the determination of the one or more physiological states of the user is based on the respiration signal and the cardiac signal, and the one or more physiological states of the user comprise a body position of the user detected based on a relative phase of the pressure signal to the acoustic signal.

18. The system of claim 17, wherein the one or more processors are further configured to detect a cardiac pressure signal of the user in the pressure signal and wherein the determination of a body position of the user is based on the cardiac pressure signal.

19. The system of claim 18, wherein the cardiac pressure signal is detected over a measurement window starting at a start measurement time and ending at an end measurement time, wherein the start measurement time is triggered by a first feature in the cardiac signal representing a start of the systolic phase of the cardiac signal and the end measurement time is triggered by a second feature in the cardiac signal representing an end of the systolic phase of the cardiac signal.

20. The system of claim 18, wherein the determination of the body position of the user is based on a phase of the cardiac pressure signal during the systolic phase of a cardiac cycle shown within the cardiac pressure signal.

21. The system of claim 20, wherein the phase of the cardiac pressure signal during the measurement window corresponds to the body position of the user such that the user lying on one side corresponds to a phase of the cardiac pressure signal where the amplitude is at a local maximum during the systolic phase and the user lying on an opposite side corresponds to another phase of the cardiac pressure signal where the amplitude is at a local minimum during the systolic phase.

22. The system of claim 17, wherein the one or more processors are further configured to output an alert if the system determines that the body position of the user has remained unchanged for a predetermined duration of time.

23. The system of claim 1, wherein the pressure signal comprises a respiration signal indicative of respiration of the user, the acoustic signal comprises a cardiac signal indicative of one or more cardiac cycles of the user, the determination of the one or more physiological states of the user is based on the respiration signal and the cardiac signal and the one or more physiological states of the user comprise a level of hydration of the user.

24. The system of claim 23, wherein the level of hydration of the user is determined by:
detecting, for each cardiac cycle in the cardiac signal, a peak of the cardiac signal across a cardiac cycle;
determining across a respiratory cycle comprising a plurality of cardiac cycles, a maximum of the peaks for the plurality of cardiac cycles and a minimum of the peaks for the plurality of cardiac cycles;
determining a difference between the maximum and the minimum;
determining an overall amplitude of the cardiac signal across the respiratory cycle; and
determining a ratio of the difference to the overall amplitude as an indicator of the level of hydration.

25. The system of claim 23, wherein the one or more processors are further configured to output an alert if the system determines that the level of hydration of the user has exceeded a predetermined hydration level threshold.

26. The system of claim 1, wherein the one or more processors are further configured to output a control signal to control an external device based on the user's physiological state, wherein the one or more physiological states comprise a level of consciousness of the user and wherein the one or more processors are configured to output the control signal in response to determining that the level of consciousness falls within a threshold range.

27. (canceled)

28. A computer-implemented method for determining one or more physiological states of a user, the method comprising:
receiving a pressure signal representing pressure within a cushioning layer supporting at least a portion of a user and an acoustic signal representing acoustic vibrations within the cushioning layer; and
determining, based on the pressure signal and acoustic signal, the one or more physiological states of the user.

29. (canceled)
